(19) United States
(12) Patent Application Publication
(10) Pub. No.: US 2003/0143666 A1
(43) Pub. Date: Jul. 31, 2003

(54) Genetic locus for everninomicin biosynthesis

(76) Inventors: Alfredo Staffa, St-Leonard (CA); Emmanuel Zazopoulos, Outremont (CA); Stephane Mercure, Verdun (CA); Piotr Peter Nowacki, Montreal (CA); Chris M. Farnet, Outremont (CA)

Correspondence Address: GEORGE A. LOUD, 3137 MOUNT VERNON AVENUE, ALEXANDRIA, VA, 22305, US

(21) Appl. No.: 09/769,734

(22) Filed: Jan. 26, 2001

Related U.S. Application Data
(60) Provisional application No. 60/177,711, filed on Jan. 27, 2000.

(51) Int. Cl. C12P 21/02; C07H 21/04; C12P 19/62; C12P 19/04; C12N 9/10; C12N 1/21; C12N 15/74

(52) U.S. Cl. 435/69.1; 435/76; 435/193; 435/252.3; 435/320.1; 536/23.2

(57) ABSTRACT

The present invention relates to isolated genetic sequences encoding proteins which direct the biosynthesis of the antibiotic everninomicin in Micromonospora carbonacea. The isolated biosynthetic gene cluster serves as a substrate for bioengineering of antibiotic structures.

### CROSS REFERENCE TO RELATED APPLICATION

This application claims benefit under 35 U.S.C. §119 of provisional application U.S. Ser. No. 60/177,170, filed on Jan. 27, 2000, which is herein incorporated by reference in its entirety for all purposes.

### FIELD OF INVENTION

The present invention relates to the field of antibiotics, specifically those active against gram-positive bacteria and more specifically to genes of the everninomicin biosynthetic pathway of Micromonospora carbonacea. In particular, this invention elucidates the gene cluster controlling the biosynthesis of everninomicin.

### BACKGROUND

Everninomicin is one member of a class of oligosaccharide natural products collectively referred to as the orthosomycins. At least five active components of everninomicin have been obtained by fermentation of M. carbonacea, namely everninomicin A, B, C, D, and E, of which everninomicin D is the principal component (Weinstein et al., Antimicrobial Agents and Chemotherapy—1964, 24-32, 1964; U.S. Pat. No. 3,499,078). Additional everninomicins, including 13-384 component 1 and 13-384 component 5, have been described from other strains of M. carbonacea (Ganguly et al., Heterocycles, 1989, Vol. 28, pp. 83-88; U.S. Pat. Nos. 4,597,968 and 4,735,903). The structure of some of the known everninomicins is described in Encyclopedia of Chemical Technology, 4^{th} edition, volume 3, 1992, pp. 60-261 ed. Mary Howe-Grant, from which the chemical structure of everninomicin, as illustrated in FIG. 2 of the present specification, was derived.

Everninomicins contain two sensitive orthoester moieties and one or more highly substituted aromatic moiteties. Everninomicins possess many unusual features, including a 1-1′ disaccharide bridge, a nitrosugar (evernitrose), thirteen rings, and thirty five stereogenic centers within its structure (Ganguly A. K. et al., Tetrahedron Lett. 1997, 38, 7989-7991). It has been recognized that everninomicin constitutes a formidable challenge to organic synthesis because of its unusual connectivity and polyfunctional and sensitive nature (Nicolaou, K. C. et al., Angew. Chem. Int. Ed. 1999, 38. No. 22). Moreover, chemical synthesis of everninomicin compounds produces a poor yield of the desired everninomicin molecule due to the presence of the unusual structural features. As an alternative to making structural analogs of microbial metabolites by chemical synthesis, manipulating genes of governing secondary metabolism offer a promising alternative and allow for preparation of these compounds biosynthetically. However, the success of a biosynthetic approach depends critically on the availability of novel genetic systems and on genes encoding novel enzyme activities. Elucidation of the everninomicin gene cluster contributes to the general field of combinatorial biosynthesis by expanding the repertoire of genes uniquely associated with everninomicin biosynthesis, leading to the making of novel everninomicins via combinatorial biosynthesis.

The emergence of multi-resistant, Gram-positive pathogens gives rise to an urgent need for new antimicrobial agents that display novel mechanisms of actions and demonstrate activity against resistant strains. Everninomicin has demonstrated a wide spectrum of antibacterial activity against gram-positive organisms, including methicillin-resistant Staphylococcus aureus, vancomycin-resistant enterococci, and penicillin-resistant pneumococci. The production of everninomicin is recognized as a valuable source of antibiotics. For example, everninomicin (trade name Ziracin®) was under development by Schering-Plough as an intravenous treatment of severe resistant gram-positive bacterial infections. Consequently, it is desirable to develop cost effective means to produce everninomicin. Elucidation of the everninomicin gene cluster would provide a means to construct everninomicin overproducing strains by de-regulating the biosynthetic machinery.

It is also desirable to produce chemical modifications of everninomicin to enhance certain properties. For example, everninomicin D presented pharmacokinetic problems when tested in vivo on mice and dogs (Ganguly A. K. et al., J. Antibiotics 35:5 561-570, 1982). Likewise, it has been reported that everninomicins have been unavailable for clinical use due to severe adverse reactions observed in laboratory animals, which reactions include lack of coordination and ataxia (Maertens, Current Opinion in Anti-infective investigational Drugs, 1999 1(1):49-56). Elucidation of the everninomicin gene cluster would provide a means to produce via genetic manipulation or combinatorial biosynthesis modified everninomicin D with improved properties. Elucidation of the gene cluster controlling the biosynthesis of everninomicin would provide access to rational engineering of everninomicin biosynthesis for novel drug leads. Accordingly, there is a need for genetic information regarding the biosynthesis of everninomicin.

### SUMMARY OF THE INVENTION

The invention provides purified and isolated polynucleotide molecules that encode polypeptides of the everninomycin biosynthetic pathway in Micromonospora carbonacea. In one form of the invention, polynucleotide molecules are selected from contiguous DNA sequences of FIG. 1 (SEQ ID NOS: 1, 3, 4, 8, 22, 36, 47 and 49). In another form, the invention provides polypeptides corresponding to the isolated DNA molecules. The amino acid sequences of the corresponding encoded polypeptides are also shown in FIG. 1.

Structural and functional characterization is provided for the 49 open reading frames (ORFs) comprising this cluster (SEQ ID NOS: 2, 5 to 7, 9 to 21, 23 to 35, 37 to 46, 48, and 50 to 58). Thus, in one embodiment, this invention provides an isolated nucleic acid comprising a nucleic acid selected from the group consisting of a nucleic acid encoding any of everninomicin ORFs 1 to 49 (SEQ ID NOS: 2, 5 to 7, 9 to 21, 23 to 35, 37 to 46, 48, and 50 to 58); a nucleic acid encoding a polypeptide encoded by any of everninomicin ORFs 1 to 49; and a nucleic acid (SEQ ID NOS: 2, 5 to 7, 9 to 21, 23 to 35, 37 to 46, 48, and 50 to 58) which is at least 75% (preferably 80%, more preferably 85% or more) identical in amino acid sequence to a polypeptide encoded by any of everninomicin ORFs 1 to 49. Certain embodiments of the invention specifically exclude one or more of ORFs 1 to 49. In one embodiment, preferred nucleic acids comprise a nucleic acid encoding at least two (more preferably at least three or more, and still more preferably at least 5 or more) ORFs selected from the group consisting of ORF 1 to 49 (SEQ ID NOS: 2, 5 to 7, 9 to 21, 23 to 35, 37 to 46, 48, and 50 to 58).

Those skilled in the art will readily understand that the invention, having provided the polynucleotide sequences encoding polypeptides of the everninomicin biosynthetic pathway, also provides polynucleotides encoding fragments derived from such peptides. In one embodiment the invention provides an isolated nucleic acid comprising a nucleic acid that specifically hybridizes under stringent conditions to an ORF of the everninomicin biosynthesis gene cluster, and can substitute for the ORF to which it specifically hybridizes to direct the synthesis of an everninomicin. In certain embodiments this also includes nucleic acids that would stringently hybridize but for the degeneracy of the nucleic acid code. In other words, if silent mutations could be made in the subject sequence so that it hybridizes to the indicated sequences under stringent conditions, it would be included in certain embodiments. The invention also provides an isolated gene cluster comprising ORFs encoding polypeptides sufficient to direct the assembly of an everninomicin or an everninomicin analogue.

Moreover, the invention is understood to provide naturally occurring variants or derivatives of such polypeptides and fragments derived therefrom, such variants or derivatives resulting from the addition, deletion, or substitution of non-essential amino acids or conservative substitutions of essential amino acids as described herein. Particularly preferred nucleic acids comprise a nucleic acid that specifically hybridizes under stringent conditions to a nucleic acid encoding a polypeptide selected from the group consisting of ORF 1, ORF 2, ORF 3, ORF 4, ORF 5, ORF 6, ORF 7, ORF 8, ORF 9, ORF 10, ORF 11, ORF 12, ORF 13, ORF 14, ORF 15, ORF 16, ORF 17, ORF 18, ORF 19, ORF20, ORF 21, ORF 22, ORF 23, ORF 24, ORF 25, ORF 26, ORF 27, ORF 28, ORF 29, ORF 30, ORF 31, ORF 32, ORF 33, ORF 34, ORF 35, ORF 36, ORF 37, ORF 38, ORF 39, ORF 40, ORF 41, ORF 42, ORF 43, ORF 44, ORF 45, ORF 46, ORF 47, ORF 48, and ORF 49 (SEQ ID NOS: 2, 5 to 7, 9 to 21, 23 to 35, 37 to 46, 48, and 50 to 58 respectively). Particularly preferred isolated nucleic acid comprises a nucleic acid encoding a polypeptide selected from the group consisting of ORF 1, ORF 2, ORF 3, ORF 4, ORF 5, ORF 6, ORF 7, ORF 8, ORF 9, ORF 10, ORF 11, ORF 12, ORF 13, ORF 14, ORF 15, ORF 16, ORF 17, ORF 18, ORF 19, ORF20, ORF 21, ORF 22, ORF 23, ORF 24, ORF 25, ORF 26, ORF 27, ORF 28, ORF 29, ORF 30, ORF 31, ORF 32, ORF 33, ORF 34, ORF 35, ORF 36, ORF 37, ORF 38, ORF 39, ORF 40, ORF 41, ORF 42, ORF 43, ORF 44, ORF 45, ORF 46, ORF 47, ORF 48, and ORF 49 (SEQ ID NOS: 2, 5 to 7, 9 to 21, 23 to 35, 37 to 46, 48, and 50 to 58 respectively). The nucleic acid may comprise a nucleic acid that is a single nucleotide polymorphism (SNP) of a nucleic acid encoding a polypeptide selected from the group consisting of ORF 1, ORF 2, ORF 3, ORF 4, ORF 5, ORF 6, ORF 7, ORF 8, ORF 9, ORF 10, ORF 11, ORF 12, ORF 13, ORF 14, ORF 15, ORF 16, ORF 17, ORF 18, ORF 19, ORF20, ORF 21, ORF 22, ORF 23, ORF 24, ORF 25, ORF 26, ORF 27, ORF 28, ORF 29, ORF 30, ORF 31, ORF 32, ORF 33, ORF 34, ORF 35, ORF 36, ORF 37, ORF 38, ORF 39, ORF 40, ORF 41, ORF 42, ORF 43, ORF 44, ORF 45, ORF 46, ORF 47, ORF 48, and ORF 49 (SEQ ID NOS: 2, 5 to 7, 9 to 21, 23 to 35, 37 to 46, 48, and 50 to 58). Certain embodiments of the invention specifically exclude one or more of ORFs 1 to 49.

This invention also provides for a polypeptide encoded by any one or more of the nucleic acids described herein.

Those skilled in the art would also readily understand that the invention, having provided the polynucleotide sequences of the entire genetic locus from M. carbonacea, further provides naturally-occurring variants or homologs of the genes of the everninomicin biosynthetic locus from other bacterial of the order Actinomycetes family. It is also understood that the invention, having provided the polynucleotide sequences of the entire genetic locus as well as the coding sequences, further provides polynucleotides which regulate the expression of the polypeptides of the biosynthetic pathway. Such regulating polynucleotides include but are not limited to promoter and enhancer sequences, as well as sequences antisense to any of the aforementioned sequences. The antisense molecules are regulators of gene expression in that they are used to suppress expression of the gene from which they are derived.

The gene cluster may be present in a host cell, preferably in a bacterial cell. Preferred families of bacterial cells include but are not limited to: a) bacteria of the family Micromonosporaceae, of which preferred genus include Micromonospora, Actinoplanes and Dactylosporangium; b) bacteria of the family Streptomycetaceae, of which preferred genus include Streptomyces, and Kitasatospora; and c) bacteria of the family Pseudonocardiaceae, of which preferred genus are Amycolatopsis, Kibdelosporangium, and Saccharopolyspora. The host cell is transformed with an exogenous nucleic acid comprising a gene cluster encoding polypeptides sufficient to direct the assembly of an everninomicin or an everninomicin analogue. In certain embodiments heterologous nucleic acid may comprise only a portion of the gene cluster, but the cell will still be able to express an everninomicin. Expression cassettes and vectors comprising a polynucleotide as described herein, as well as cells transformed or transfected with such cassettes and vectors, are also within the scope of the invention.

The invention also provides methods of chemically modifying a biological molecule. The methods involve contacting a biological molecule that is a substrate for a polypeptide encoded by an everninomicin biosynthesis gene cluster ORF, with a polypeptide encoded by an everninomicin biosynthesis gene cluster ORF whereby the polypeptide chemically modifies the biological molecule. In one preferred embodiment, the polypeptide is an enzyme selected from the group consisting of an O-methyltransferase, an integral membrane antiporter, a methyltransferase, a blue copper oxidoreductase, a C-methyltransferase, a nucleotide binding protein, a mannosyltransferase, a sugar epimerase/reductase, an oxygenase, a tRNA/rRNA methylase, a 3-ketoacyl-[ACP]-synthase, a glycosyltransferase, an alpha-ketoglutarate-dependent dioxygenase, a halogenase, a glycosyltransferase, an acetoin dehydrogenase E1 alpha or beta subunit, a rhamnosyltransferase, a sugar dehydratase/epimerase, a sugar nucleotidyltransferase, a sugar 4,6-dehydratase, a sugar epimerase/ketoreductase, an iterative type 1 polyketide synthase, a hydrolase/phosphatase, a glucosyltransferase, a sugar ketoreductase, sugar 2,3-dehydratase, sugar dehydratase, a resistance rRNA methyltransferase, a flavoprotein oxidoreductase, a deoxyhexose aminotransferase, a sugar epimerase, a sugar ketoreductase, an endoglucanase, a transcriptional regulator and a glucokinase. In a preferred embodiment, the method involves contacting the biological molecule with at least two (preferably at least three or more) different polypeptides of everninomicin gene cluster ORFs 1 to 49 (SEQ ID NOS: 2, 5 to 7, 9 to 21, 23 to 35, 37 to 46, 48, and 50 to 58). The contacting may be in a host cell or the contacting can be ex vivo. The biological molecule can be an endogenous metabolite produced by the host cell or an exogenous supplied metabolite. In preferred embodiments, the host cell is a bacterial cell or eukaryotic cell (e.g. a mammalian cell, a yeast cell, a plant cell, a fungal cell, an insect cell etc.). In certain preferred embodiments, the host cell synthesizes deoxyhexose precursors or a dichloroisoeverninic moiety for the biological molecule. In other preferred embodiments, the host cell synthesizes the nitrosugar evernitrose. In one preferred embodiment, the method comprises contacting the biological molecule with substantially all of the polypeptides of ORF 1 to 49 (SEQ ID NOS: 2, 5 to 7, 9 to 21, 23 to 35, 37 to 46, 48, and 50 to 58) and the method produces an everninomicin or everninomicin analogue.

### BRIEF DESCRIPTION OF THE DRAWINGS

FIG. 1 illustrates contiguous nucleotide sequences and deduced amino acid sequences of the everninomicin biosynthetic locus from Micromonospora carbonacea (SEQ ID NOS: 1 to 58).

FIG. 2 illustrates the structure of some of the known everninomicins.

FIG. 3 illustrates a biosynthetic scheme for the production of deoxyhexose precursors for everninomicin biosynthesis.

FIG. 4 illustrates a biosynthetic scheme for the production of nitrosugar evernitrose.

FIG. 5 illustrates a biosynthetic scheme for the production of the dichloroisoeverninic moiety that is found in the ester linkage to the sugar residue B of everninomicin.

### DETAILED DESCRIPTION OF THE INVENTION

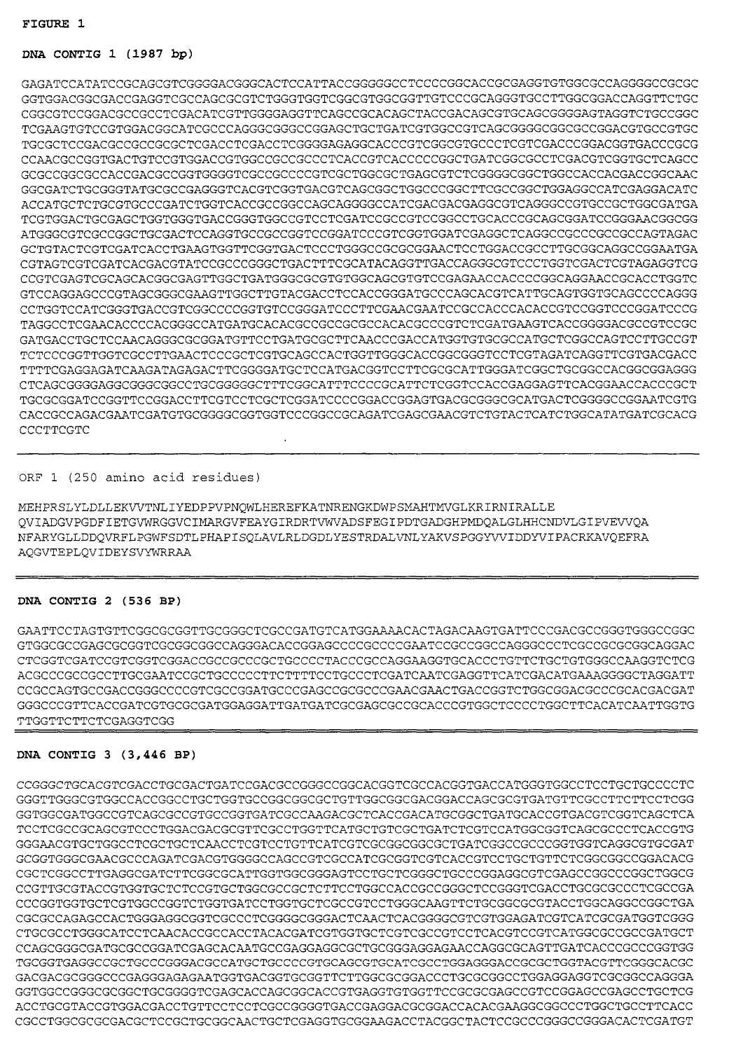

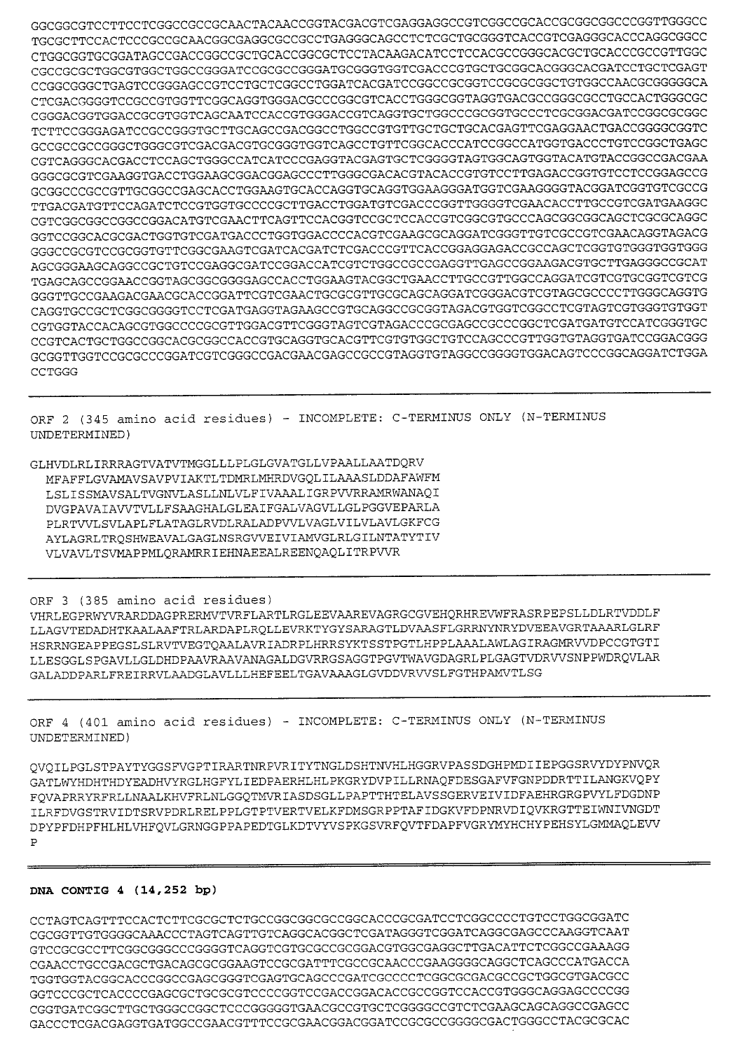

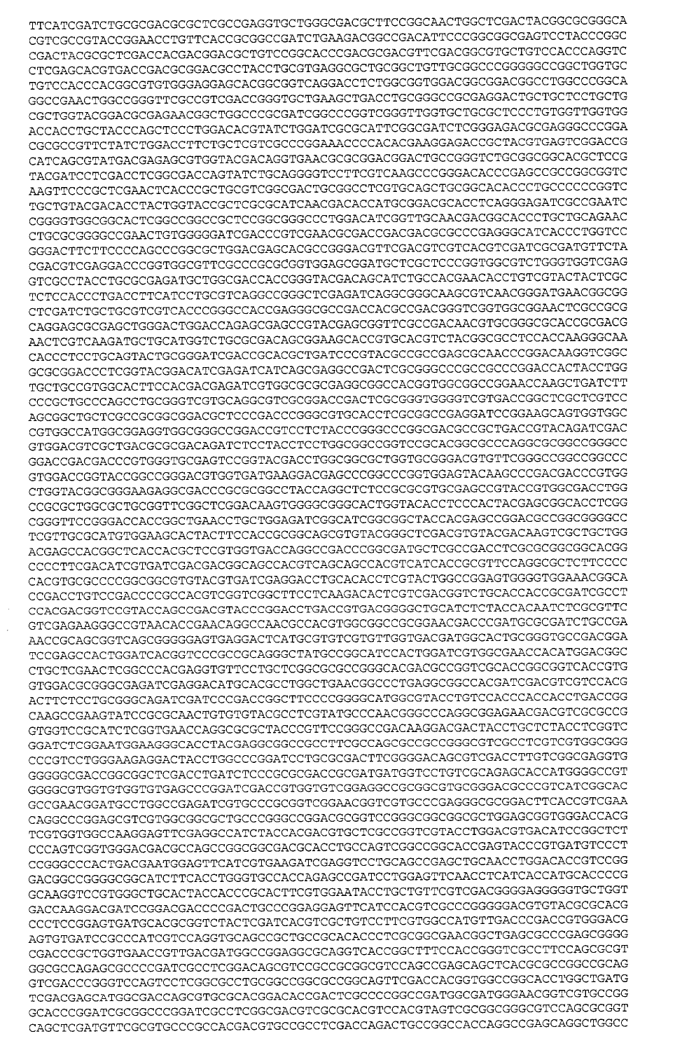

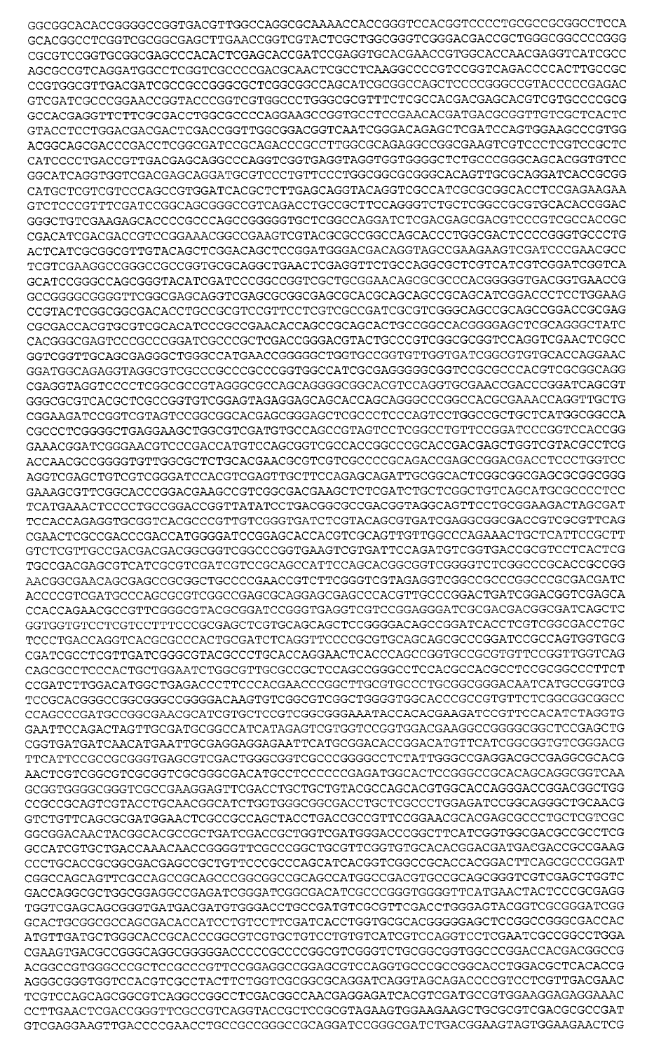

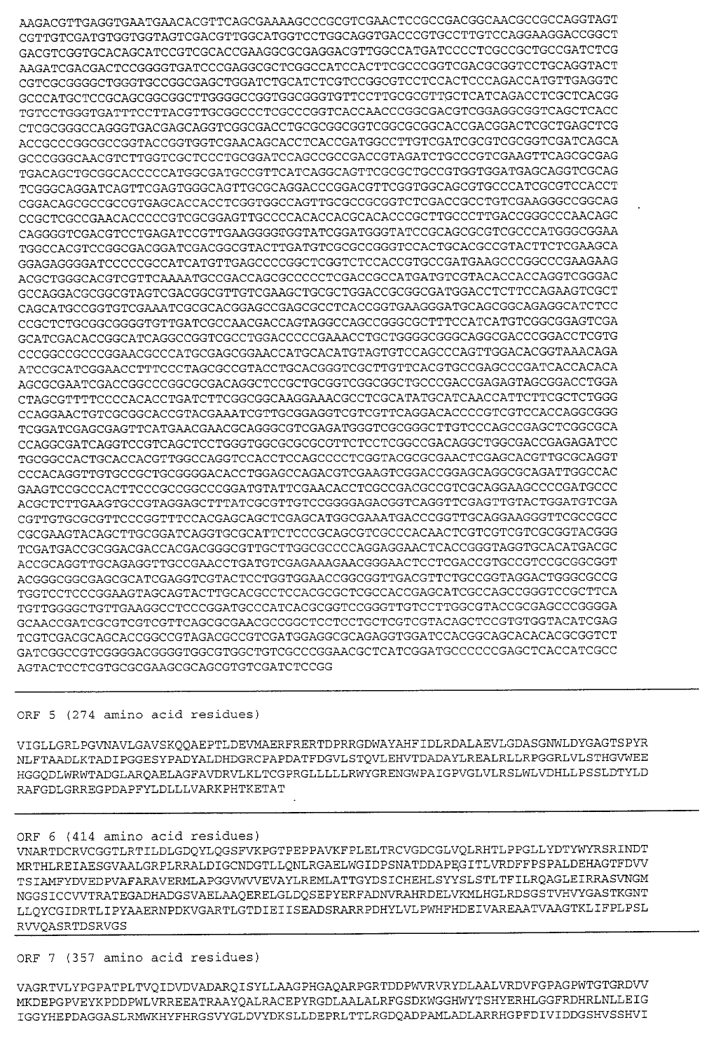

Contiguous nucleotide sequences and deduced amino acid sequences of the everninomicin biosynthetic locus from Micromonospora carbonacea are illustrated in FIG. 1 (SEQ ID NOS: 1 to 58). In particular, FIG. 1 shows a complete gene cluster formed of eight DNA contiguous sequences, which gene cluster regulates the biosynthesis of everninomicin. FIG. 1 further shows the amino acid sequences of the isolated polynucleotide coding regions which encode 49 polypeptides of the everninomicin biosynthetic pathway (SEQ ID NOS: 2, 5 to 7, 9 to 21, 23 to 35, 37 to 46, 48, and 50 to 58).

The contiguous nucleotide sequences are arranged such that, as found within the everninomicin biosynthetic locus, DNA contig 1 (SEQ ID NO 1) is adjacent to the 5′ end of DNA contig 2 (SEQ ID NO 3), which is in turn adjacent to DNA contig 3 (SEQ ID NO 4), etc. The ORFs represent open reading frames deduced from the nucleotide sequences. ORF 1 (SEQ ID NO 2) has been deduced from DNA contig 1 (SEQ ID NO 1); ORFs 2 to 4 (SEQ ID NOS: 3, 4, and 8) have been deduced from DNA contig 3 (SEQ ID NO 4); ORFs 5 to 17 (SEQ ID NOS: 9 to 21) have been deduced from DNA contig 4 (SEQ ID NO 8); ORFs 18 to 30 (SEQ ID NOS: 23 to 35) have been deduced from DNA contig 5 (SEQ ID NO 22); ORFs 31 to 39 (SEQ ID NOS 37 to 45) and the C-terminus of ORF 40 (SEQ ID NO 46) have been deduced from DNA contig 6 (SEQ ID NO 36); the N-terminus of ORF 40 (SEQ ID NO 48) has been deduced from DNA contig 7 (SEQ ID NO 47); ORFs 41 to 49 (SEQ ID NOS 50 to 58) have been deduced from DNA contig 8 (SEQ ID NO 49). As pointed out in FIG. 1, some of the ORFs are incomplete. In addition, one nucleotide (at position 27 of DNA contig 6, SEQ ID NO 36) remains to be determined. The DNA contig coding regions giving rise to the ORFs are also shown in FIG. 1, along with the orientation of the ORFs, (i.e. whether they are to be read off the positive (sense, coding) strand or the negative (antisense, non-coding strand)).

A deposit of three strains of E.coli DH10B cells, each harbouring a cosmid clone of the everninomicin locus was made on Jan. 24, 2001 with the International Depositary Authority of Canada (IDAC), 1015 Arlington Street, Winnipeg, Manitoba, R3E 3R2, Canada according to the provisions of the Budapest Treaty. The deposits were assigned accession nos. IDAC 240101-1, IDAC 240101-2 and IDAC 240101-3. All restrictions on the availability to the public of the above IDAC deposits will be irrevocably removed upon the granting of a patent on this application.

Everninomicin is naturally produced by a number of microorganisms of the order Actinomycetales. Given the potential medical importance of this class of antibiotics, the genetic locus encoding the biosynthetic pathway for everninomicin production was isolated and sequenced from one known producer, Micromonospora carbonacea subspecies aurantiaca (strain number NRRL 2997, obtained from the Agricultural Research Service Culture Collection of the United States Department of Agriculture; everninomicin production by this strain is described in U.S. Pat. No. 3,499,078). The newly discovered locus encodes 49 individual proteins (SEQ ID NOS: 2, 5 to 7, 9 to 21, 23 to 35, 37 to 46, 48, and 50 to 58) involved in the biosynthesis of everninomicin by this organism. The full-length locus and individual cloned genes are useful for a variety of purposes relating to synthesis of antibiotics of the orthosomycin class.

The entire everninomycin biosynthetic locus spans approximately 60 kb. Analysis of this 60 kb DNA sequence reveals the presence of individual genes encoding 49 individual proteins. Three of the genes show strong homology to the Streptomyces viridochromogenes avilamycin biosynthetic genes aviD, aviE and aviM, previously demonstrated to be involved in the biosynthesis of avilamycin, a member of the orthosomycin class of antibiotics (Gaisser et al., 1997, J. Bacteriol., Vol. 179, pp. 6271-6278). The gene encoding ORF 28 of FIG. 1 (SEQ ID NO 33) is homologous to the aviD gene, the gene encoding ORF 29 of FIG. 1 (SEQ ID NO 34) is homologous to the aviE gene, and the gene encoding ORF 32 of FIG. 1 (SEQ ID NO 38) is homologous to the aviM gene.

The functions of the 49 individual proteins of the everninomicin biosynthetic locus were assessed by computer comparison of each protein with proteins found in the GenBank database of protein sequences (National Center for Biotechnology Information, National Library of Medicine, Bethesda, Md. USA) using the BLASTP algorithm (Altschul et al., 1997, Nucleic Acids Res. Vol. 25, pp.3389-3402). Significant amino acid sequence homologies and proposed function found for each protein in the everninomicin locus are shown in Table 1.

TABLE 1

| | | | GenBank | | % | % | |
|---|---|---|---|---|---|---|---|
| ORF | # aa | Proposed function | homology | probability | identity | similarity | proposed function of GenBank match |
| 1 | 250 | O-methyltransferase | AAD41819 | 5.00E−83 | 55 | 71 | TylF 3″″-O-methyltransferase in tylosin |
| | | | | | | | biosynthetic locus of Streptomyces fradiae |
| | | | BAA03670 | 3.00E−80 | 54 | 71 | MycF mycinamicin III O-methyltransferase |
| | | | | | | | in the mycinamicin biosynthetic locus of |
| | | | AAG29794 | 1.00E−79 | 56 | 70 | Micromonospora griseorubida |
| | | | | | | | CumN O-methyltransferase in coumermycin |
| | | | AAF67509 | 2.00E−79 | 56 | 70 | A1 biosynthetic locus of Streptomyces rishiriensis |
| | | | | | | | NovP O-methyltransferase in the novobiocin |
| | | | | | | | biosynthetic locus of Streptomyces spheroides |
| 2 | 345 | integral membrane | AAF26906 | 6.00E−38 | 31 | 48 | protein similar to Na/H and drug/H antiporters |
| | | antiporter | | | | | in epothilone biosynthetic locus of |
| | | | | | | | Sorangium cellulosum |
| | (partial) | | CAB45049 | 2.00E−35 | 31 | 54 | putative integral membrane ion antiporter in |
| | | | | | | | chloroeremomycin biosynthetic locus of |
| | | | | | | | Amycolatopsis orientalis |
| | | | BAA16991 | 6.00E−33 | 26 | 49 | Synechocystis sp. Na/H antiporter |
| 3 | 385 | methyltransferase | BAA79525 | 6.00E−15 | 28 | 41 | hypothetical protein in Aeropyrum pemix with |
| | | | | | | | homology to N-6 Adenine-specific DNA methylases |
| | | | CAB88946 | 6.00E−05 | 31 | 40 | putative methyltransferase in Streptomyces coelicolor |
| 4 | 480 | blue copper | CAB12449 | 1.00E−60 | 33 | 44 | Bacillus subtilis spore coat protein involved |
| | | oxidoreductase | | | | | in brown pigmentation during sporogenesis |
| | (partial) | | BAA02123 | 6.00E−60 | 35 | 49 | bilirubin oxidase from Myrothecium verrucaria |
| | | | CAB75422 | 7.00E−57 | 34 | 47 | polyphenol oxidase from Acremonium morurum |
| | | | AAA86668 | 3.00E−35 | 26 | 37 | PhsA phenoxazinone synthase from Streptomyces |
| | | | | | | | Antibioticus |
| 5 | 274 | methyltransferase | AAF09939 | 9.00E−05 | 53 | 64 | probable methyltransferase, BioC family, from |
| | | | | | | | Deinococcus radiodurans |
| | | | AAC01738 | 7.00E−05 | 35 | 45 | methyltransferase in rifamycin biosynthetic locus |
| | | | | | | | of Amycolatopsis mediterranei |
| | | | CAB93437 | 3.00E−04 | 42 | 70 | putative methyltransferase from |
| | | | | | | | Streptomyces coelicolor |
| 6 | 414 | C-methyltransferase | AAD41823 | 4.00E−79 | 43 | 55 | TylCIII NDP-hexose 3-C-methyltransferase |
| | | | | | | | in thetylosin biosynthetic locus of |
| | | | | | | | Streptomyces fradiae |
| | | | CAA42926 | 4.00E−72 | 41 | 55 | protein in the erythromycin biosynthetic locus |
| | | | | | | | of Saccharopolyspora erythraea |
| | | | AAG29803 | 5.00E−46 | 31 | 49 | CumW C-methyltransferase in the coumermycin |
| | | | | | | | A1 biosynthetic locus of Streptomyces rishiriensis |
| | | | AAF01816 | 1.00E−45 | 31 | 47 | SnoG protein in the nogalamycin biosynthetic |
| | | | | | | | locus of Streptomyces nogalater |
| | | | AAF67514 | 6.00E−44 | 30 | 47 | NovU C-methyltransferase in the novobiocin |
| | | | | | | | biosynthetic locus of Streptomyces spheroides |
| 7 | 357 | O-methyltransferase | AAD12164 | 3.00E−79 | 45 | 59 | TylE O-methyltransferase in the tylosin biosynthetic |
| | | | | | | | locus of Streptomyces fradiae |
| | | | CAA12021 | 6.00E−72 | 45 | 57 | SnogY O-methylase in the nogalamycin biosynthetic |
| | | | | | | | locus of Streptomyces nogalater |
| | | | CAA05644 | 7.00E−52 | 42 | 56 | OleY protein in the oleandomycin biosynthetic |
| | | | | | | | locus of Streptomyces antibioticus |
| 8 | 292 | mannosyltransferase | AAB89517 | 1.00E−05 | 26 | 47 | galactosyltransferase from Archaeoglobus fulgidus |
| | | | CAB58332 | 6.00E−05 | 26 | 38 | putative glycosyl transferase from Streptomyces |
| | | | | | | | coelicolor |
| | | | AAF12269 | 3.00E−04 | 25 | 45 | mannosyl transferase from Deinococcus |
| | | | | | | | radiodurans |
| 9 | 137 | nucleotide-binding | AAD45266 | 3.60E+00 | 34 | 42 | Pseudomonas aeruginosa WbjC putative |
| | | protein | | | | | nucleotide-binding protein involved in O-antigen |
| | | | | | | | (sugar) biosynthesis |
| | | | AAB63947 | 6.20E+00 | 38 | 60 | Streptococcus pneumoniae SulD bifunctional |
| | | | | | | | aldolase-pyrophosphokinase |
| 10 | 314 | sugar epimerase/reductase | CAA12010 | 1.00E−51 | 42 | 53 | SnogG dTD P-4-keto-6-deoxyhexose reductase |
| | | | | | | | in the nogalamycin biosynthetic locus of Streptomyces |
| | | | | | | | nogalater |
| | | | AAB63047 | 4.00E−46 | 38 | 52 | DnmV thymidine diphospho-4-keto-2,3,6- |
| | | | | | | | trideoxyhexulose reductase in the daunorubicin |
| | | | | | | | biosynthetic locus of Streptomyces peucetius |
| | | | AAD13561 | 5.00E−45 | 39 | 50 | LanZ3 NDP-hexose 4-keto reductase in the |
| | | | | | | | landomycin biosynthetic locus of |
| | | | | | | | Streptomyces cyanogenus |
| | | | AAF72549 | 4.00E−43 | 39 | 48 | UrdZ3 NDP-hexose 4-ketoreductase in the urdamycin |
| | | | | | | | biosynthetic of Streptomyces fradiae |
| 11 | 285 | O-methyltransferase | BAA32132 | 2.00E−68 | 50 | 61 | methyltransferase in Streptomyces griseus |
| | | | AAB00531 | 2.00E−63 | 46 | 59 | DmpM O-demethylpuromycin-O-methyltransferase |
| | | | | | | | in the puromycin biosynthetic locus of Streptomyces |
| | | | | | | | alboniger |
| | | | AAD32742 | 8.00E−34 | 34 | 47 | MmcR O-methyltransferase in the mitomycin |
| | | | | | | | biosynthetic locus of Streptomyces lavendulae |
| | | | AAA67518 | 4.00E−32 | 33 | 48 | TcmN O-methyltransferase in the tetracenomycin |
| | | | | | | | biosynthetic locus of Streptomyces glaucescens |
| 12 | 276 | Oxygenase | CAA07766 | 5.00E+00 | 27 | 39 | MtmOl oxygenase in the mithramycin biosynthetic |
| | | | | | | | locus of Streptomyces argillaceus |
| 13 | 265 | tRNA/rRNA methylase | AAG32066 | 3.00E−73 | 54 | 70 | rRNA methyltransferase AviRb involved in avilamycin |
| | | | | | | | A resistance Streptomyces viridochromogenes |
| | | | AAF10591 | 7.00E−28 | 36 | 51 | rRNA methylase from Deinococcus radiodurans |
| | | | AAF73591 | 1.00E−23 | 31 | 48 | SpoU rRNA methylase family protein from |
| | | | | | | | Chlamydia muridarum |
| | | | AAC68000 | 1.00E−22 | 30 | 48 | SpoU family rRNA methylase from Chlamydia |
| | | | | | | | Trachomatis |
| | | | AAD18670 | 2.00E−22 | 27 | 48 | SpoU-1 rRNA methylase fromChlamydophila |
| | | | | | | | pneumoniae |
| 14 | 344 | 3-ketoacyl-[ACP]-synthase | AAG29787 | 2.00E−76 | 43 | 58 | CumJ 3-ketoacyl-[ACP]-synthase in the coumermycin |
| | | | | | | | A1 biosynthetic locus of Streptomyces rishiriensis |
| | | | AAA65208 | 2.00E−61 | 38 | 54 | DpsC daunorubicin-doxorubicin polyketide synthase |
| | | | | | | | from Streptomyces peucetius |
| | | | CAB71914 | 3.00E−70 | 40 | 58 | beta-keto acyl synthase III homolog form Streptomyces |
| | | | | | | | coelicolor |
| | | | AAF70109 | 5.00E−54 | 37 | 50 | AknE2 ketoacyl synthase involved in aclacinomycin |
| | | | | | | | biosynthesis in Streptomyces galilaeus |
| 15 | 240 | methyltransferase | CAA70016 | 5.00E−04 | 33 | 41 | StsG methyltransferase involved in N-methyl-L- |
| | | | | | | | glucosamine pathway in streptomycin biosynthetic |
| | | | | | | | locus of Streptomyces griseus |
| | | | AAG06559 | 2.00E−03 | 24 | 41 | UbiG 3-demethylubiquinone-9 3-methyltransferase |
| | | | | | | | from Pseudomonas aeruginosa |
| | | | AAF09618 | 5.00E−03 | 27 | 47 | putative methyltransferase from |
| | | | | | | | Deinococcus radiodurans |
| | | | AAD28458 | 1.50E−02 | 27 | 43 | MitN methyltransferase in the mitomycin biosynthetic |
| | | | | | | | locus of Streptomyces lavendulae |
| 16 | 380 | glycosyltransferase | AAF00209 | 5.00E−80 | 44 | 58 | UrdGT2 glycosyl transferase in the urdamycin |
| | | | | | | | A biosynthetic locus of Streptomyces fradiae |
| | | | AAD13553 | 7.00E−78 | 43 | 59 | LanGT2 glycosyl transferase in the landomycin |
| | | | | | | | biosynthetic locus of Streptomyces cyanogenus |
| | | | CAA09635 | 8.00E−70 | 42 | 55 | Gra-orf14 putative glycosyl transferase in the |
| | | | | | | | granaticin biosynthetic locus of Streptomyces |
| | | | | | | | violaceoruber |
| | | | AAC01731 | 3.00E−58 | 37 | 51 | dNTP-hexose glycosyl transferase in the rifamycin |
| | | | | | | | biosynthetic locus of Amycolatopsis mediterranei |
| 17 | 405 | unknown | none | | | | |
| 18 | 296* | alpha-ketoglutarate- | AAC71711 | 0.005 | 27 | 42 | HtxA putative alpha-ketoglutarate-dependent |
| | (partial) | dependent | | | | | Hypophosphite dioxygenase from |
| | | dioxygenase | | | | | Pseudomonas stutzeri |
| 19 | 243 | methyltransferase | JC5319 | 9.90E−02 | 43 | 61 | TlrD macrolide-lincosamide-streptogramin B |
| | | | | | | | resistance determinant from Streptomyces fradiae |
| | | | CAB45043 | 2.20E−01 | 36 | 49 | putative rRNA methylase from Amycolatopsis |
| | | | | | | | orientalis |
| | | | AAF86398 | 3.80E−01 | 26 | 35 | FkbM 31-O-methyltransferase in the FK520 |
| | | | | | | | biosynthetic locus of Streptomyces |
| | | | | | | | hygroscopicus var. ascomyceticus |
| | | | AAC44360 | 3.80E−01 | 30 | 40 | FkbM 31-O-demethyl-FK506 methyltransferase |
| | | | | | | | in the FK506 biosynthetic locus of Streptomyces sp. |
| 20 | 482 | halogenase | CAA11780 | 6.00E−60 | 32 | 50 | protein similar to non-heme oxygenase/halogenase |
| | | | | | | | in chloroeremomycin biosynthetic locus of |
| | | | | | | | Amycolatopsis orientalis |
| | | | CAA76550 | 5.00E−59 | 32 | 49 | OxyD putative halogenase in the balhimycin |
| | | | | | | | biosynthetic locus of Amycolatopsis mediterranei |
| | | | AAG38844 | 2.00E−34 | 31 | 47 | putative reductase/halogenase in the xanthomonadin |
| | | | | | | | biosynthetic locus of Xanthomonas oryzae |
| | | | AAD24884 | 7.00E−29 | 27 | 43 | PltA putative halogenase in the pyoluteorin |
| | | | | | | | biosynthetic locus of Pseudomonas fluorescens |
| 21 | 438 | glycosyltransferase | AAC64928 | 2.00E−44 | 32 | 44 | MtmGI glycosyltransferase involved in mithramycin |
| | | | | | | | biosynthesis in Streptomyces argillaceus |
| | | | AAD55583 | 2.00E−43 | 32 | 46 | MtmGIII glycosyltransferase involved in mithramycin |
| | | | | | | | biosynthesis in Streptomyces argillaceus |
| | | | AF077869 | 2.00E−41 | 32 | 44 | MtmGIV glycosyltransferase involved in mithramycin |
| | | | | | | | biosynthesis in Streptomyces argillaceus |
| | | | AAC68677 | 3.00E−34 | 28 | 42 | DesVII glycosyl transferase in the |
| | | | | | | | methymycin/pikromycin |
| | | | | | | | biosynthetic locus of Streptomyces venezuelae |
| 22 | 325 | acetoin dehydrogenase | AAG07537 | 8.00E−71 | 48 | 60 | probable dehydrogenase E1 component from |
| | | E1 alpha subunit | | | | | Pseudomonas aeruginosa |
| | | | AAA21744 | 8.00E−69 | 46 | 61 | TPP-dependent acetoin dehydrogenase E1 alpha- |
| | | | | | | | subunit from Clostridium magnum |
| | | | AAA21948 | 3.00E−65 | 46 | 57 | Acetoin:DCPIP oxidoreductase-alpha from Ralstonia |
| | | | | | | | eutropha |
| 23 | 320 | acetoin dehydrogenase | AAA18916 | 2.00E−53 | 38 | 55 | Acetoin:DCPIP oxidoreductase beta subunit from |
| | | E1 beta subunit | | | | | Pelobacter carbinolicus |
| | | | AAG07538 | 8.00E−53 | 40 | 54 | Acetoin catabolism protein AcoB from Pseudomonas |
| | | | | | | | aeruginosa |
| | | | AAA21745 | 6.00E−52 | 37 | 57 | TPP-dependent acetoin dehydrogenase beta-subunit |
| | | | | | | | from Clostridium magnum |
| 24 | 337 | Rhamnosyltransferase | CAB50099 | 2.00E−18 | 31 | 48 | rhamnosyl transferase related protein from |
| | | | | | | | Pyrococcus abyssi |
| | | | AAF04375 | 5.00E−18 | 29 | 42 | WbbL dTDP-Rha:a-D-GlcNAc-diphosphoryl |
| | | | | | | | polyprenol a-3-L-rhamnosyl transferase from |
| | | | | | | | Mycobacterium smegmatis |
| | | | AAF12271 | 3.00E−16 | 27 | 45 | putative rhamnosyltransferase from Deinococcus |
| | | | | | | | radiodurans |
| | | | AAB66522 | 2.00E−15 | 24 | 44 | putative rhamnosyl transferase involved in capsular |
| | | | | | | | polysaccharide biosynthesis in Streptococcus |
| | | | | | | | pneumoniae |
| 25 | 350 | unknown | None | | | | |
| 26 | 252 | alpha-ketoglutarate- | AAF01812 | 1.00E−12 | 28 | 41 | SnoK protein in the nogalamycin biosynthetic locus of |
| | | dependent dioxygenase | | | | | Streptomyces nogalater |
| | | | AAC71711 | 3.00E−11 | 23 | 42 | HtxA putative alpha-ketoglutarate-dependent |
| | | | | | | | hypophosphite dioxygenase from Pseudomonas |
| | | | | | | | stutzeri |
| | | | AAB81835 | 3.00E−06 | 23 | 35 | peroxisomal phytanoyl-CoA alpha-hydroxylase from |
| | | | | | | | Mus musculus |
| | | | AAF15971 | 2.00E−05 | 23 | 38 | 2-oxoglutarate dependent peroxisomal phytanoyl-CoA |
| | | | | | | | hydroxylase (dioxygenase) from Rattus norvegicus |
| 27 | 309 | sugar dehydratase/ | AAG08838 | 4.00E−46 | 38 | 53 | Gmd GDP-mannose 4,6-dehydratase from |
| | | epimerase | | | | | Pseudomonas aeruginosa |
| | | | AAC38668 | 7.00E−46 | 37 | 51 | LpsA putative GDP-mannose-4,6-dehydratase |
| | | | | | | | predicted to be involved in S-layer lipopolysaccharide |
| | | | | | | | biosynthesis in Caulobacter crescentus |
| | | | AAC44117 | 6.00E−44 | 37 | 51 | Gca GDP-D-mannose dehydratase involved in |
| | | | | | | | common antigen biosynthesis in Pseudomonas |
| | | | | | | | aeruginosa |
| | | | AAB84839 | 7.00E−43 | 34 | 50 | GDP-D-mannose dehydratase in |
| | | | | | | | Methanothermobacter thermoautotrophicus |
| | | | AAD20373 | 2.00E−42 | 36 | 50 | MdhtA GDP-D-mannose-dehydratase found in |
| | | | | | | | glycopeptolipid biosynthetic locus of Mycobacterium |
| | | | | | | | avium |
| 28 | 355 | Sugar | P08075 | 1.00E−126 | 61 | 77 | StrD glucose-1-phosphate thymidylyltransferase found |
| | | nucleotidyltransferase | | | | | in the streptomycin biosynthetic locus in Streptomyces |
| | | | | | | | griseus |
| | | | T30872 | 1.00E−125 | 60 | 78 | AviD dNDP-glucose synthase in the avilamycin |
| | | | | | | | biosynthetic locus of Streptomyces viridochromogenes |
| | | | AAD28517 | 1.00E−124 | 59 | 77 | BlmD streptomycin strD protein homolog in the |
| | | | | | | | bluensomycin biosynthetic locus of Streptomyces |
| | | | | | | | bluensis |
| | | | T48866 | 1.00E−123 | 60 | 77 | MtmD glucose-1-phosphate thymidylyltransferase in |
| | | | | | | | the mithramycin biosynthetic locus of Streptomyces |
| | | | | | | | argillaceus |
| 29 | 329 | sugar 4,6-dehydratase | T30873 | 1.00E−139 | 74 | 82 | AviE dNDP-glucose dehydratase in the avilamycin |
| | | | | | | | biosynthetic locus of Streptomyces viridochromogenes |
| | | | AAG18457 | 1.00E−123 | 66 | 75 | AprE dTDP-glucose 4,6-dehydratase from |
| | | | | | | | Streptomyces tenebrarius |
| | | | AAA68211 | 1.00E−123 | 66 | 75 | TDP-D-glucose-4,6-dehydratase in the erythromycin |
| | | | | | | | biosynthetic locus of Saccharopolyspora erythraea |
| | | | BAA84593 | 1.00E−115 | 63 | 76 | AveBII dTDP-glucose 4,6-dehydratase in the |
| | | | | | | | avermectin biosynthetic locus of Streptomyces |
| | | | | | | | avermitilis |
| | | | AAC68681 | 1.00E−114 | 62 | 74 | DesIV TDP-glucose-4,6-dehydratase in the |
| | | | | | | | methymycin/pikromycin biosynthetic locus of |
| | | | | | | | Streptomyces venezuelae |
| 30 | 342 | sugar epimerase/ | AAD35594 | 6.00E−43 | 38 | 53 | UDP-glucose 4-epimerase from Thermotoga maritima |
| | | ketoreductase | | | | | |
| | | | AAG07455 | 3.00E−37 | 37 | 51 | probable epimerase from Pseudomonas aeruginosa |
| | | | A71183 | 2.00E−34 | 33 | 46 | probable UDP-glucose 4-epimerase from Pyrococcus |
| | | | | | | | horikoshii |
| | | | CAB49227 | 1.00E−33 | 33 | 46 | GalE-1 UDP-glucose 4-epimerase from Pyrococcus |
| | | | | | | | abyssi |
| 31 | 354 | alpha-ketoglutarate- | AAF01812 | 1.00E−10 | 26 | 41 | Snok protein in the nogalamycin biosynthetic locus of |
| | | dependent dioxygenase | | | | | Streptomyces nogalater |
| | | | AAB81835 | 3.00E−07 | 29 | 43 | peroxisomal phytanoyl-CoA alpha-hydroxylase from |
| | | | | | | | Mus musculus |
| | | | AAC71711 | 4.00E−06 | 25 | 41 | HtxA putative alpha-ketoglutarate-dependent |
| | | | | | | | hypophosphite dioxygenase from Pseudomonas |
| | | | | | | | stutzeri |
| 32 | 1267 | iterative type I | CAA72713 | 0.00E+00 | 65 | 75 | AviM orsellinic acid synthase in the avilamycin |
| | | polyketide synthase | | | | | biosynthetic locus of Streptomyces viridochromogenes |
| | | | BAA20102 | 0.00E+00 | 40 | 56 | 6-methylsalicylic acid synthase from Aspergillus |
| | | | | | | | terreus |
| | | | S13178 | 0.00E+00 | 41 | 55 | 6-methylsalicylic acid synthase from Penicillium |
| | | | | | | | griseofulvum |
| 33 | 303 | hydrolase/ | AAF09992 | 1.00E−05 | 31 | 43 | hydrolase of the CbbY/CbbZ/GpH/YieH family from |
| | | phosphatase | | | | | Deinococcus radiodurans |
| | | | AAG19324 | 1.00E−05 | 32 | 46 | p-nitrophenyl phosphatase from Halobacterium sp. |
| | | | AAC76410 | 4.00E−03 | 33 | 53 | phosphoglycolate phosphatase from Escherichia coli |
| 34 | 307 | sugar epimerase/ | AAD45554 | 2.00E−52 | 43 | 55 | Spcl putative dNDP-glucose-4,6-dehydratase in the |
| | | ketoreductase | | | | | spectinomycin biosynthetic locus of Streptomyces |
| | | | | | | | flavopersicus |
| | | | CAA18814 | 1.00E−23 | 32 | 43 | putative sugar dehydratase from Mycobacterium |
| | | | | | | | leprae |
| | | | AAD35594 | 2.00E−23 | 28 | 44 | UDP-glucose 4-epimerase from Thermotoga maritima |
| | | | BAA84595 | 2.00E−17 | 30 | 42 | AviBIV dTDP-4-keto-6-deoxy-L-hexose 4-reductase in |
| | | | | | | | the avermectin biosynthetic locus of Streptomyces |
| | | | | | | | avermitilis |
| 35 | 295 | glycosyltransferase | S37028 | 6.00E−05 | 28 | 42 | ExoM rhizobium succinoglycan biosynthesis |
| | | | | | | | glycosyltransferase from Sinorhizobium meliloti |
| | | | AAB90621 | 2.20E−01 | 25 | 42 | ExoM succinoglycan biosynthesis protein from |
| | | | | | | | Archaeoglobus fulgidus |
| 36 | 341 | sugar ketoreductase | AAF73453 | 6.00E−91 | 55 | 69 | AknQ putative 3-ketoreductase in the Streptomyces |
| | | | | | | | galilaeus aclacinomycin biosynthetic locus |
| | | | AAD13550 | 2.00E−87 | 53 | 65 | LanT oxidoreductase homolog found in the |
| | | | | | | | landomycin biosynthetic locus of Streptomyces |
| | | | | | | | cyanogenus |
| | | | AAA83425 | 3.00E−85 | 48 | 64 | RdmF oxidoreductase of Streptomyces purpurascens |
| | | | AAF59931 | 4.00E−82 | 50 | 65 | dTDP-3,4-diketo-2,6-dideoxyglucose 3-ketoreductase |
| | | | | | | | involved in the 2-deoxygenation step in dTDP-L- |
| | | | | | | | oleandrose biosynthesis |
| 37 | 470 | sugar 2,3-dehydratase | AAD55451 | 1.00E−127 | 52 | 64 | OleV involved in the C-2 deoxygenation step in |
| | | | | | | | dTDP-L-oleandrose biosynthesis in |
| | | | | | | | Streptomyces antibioticus |
| | | | CAB96551 | 1.00E−122 | 52 | 63 | MtmV D-olivose, D-oliose and D-mycarose 2,3- |
| | | | | | | | dehydratase in the mithramycin biosynthetic locus |
| | | | | | | | of Streptomyces argillaceus |
| | | | T46668 | 1.00E−119 | 51 | 64 | SnogH probable 2,3-dehydratase in the nogalamycin |
| | | | | | | | biosynthetic locus of |
| | | | | | | | Streptomyces nogalater |
| | | | AAD13549 | 1.00E−118 | 50 | 63 | LanS NDP-hexose 2,3-dehydratase homolog in the |
| | | | | | | | landomycin biosynthetic locus of |
| | | | | | | | Streptomyces cyanogenus |
| 38 | 346 | sugar dehydratase | AAF71765 | 1.00E−120 | 63 | 77 | NysDIII putative dGDP-mannose-4,6-dehydratase in |
| | | | | | | | the nystatin biosynthetic locus of |
| | | | | | | | Streptomyces noursei |
| | | | AAG35360 | 4.00E−96 | 55 | 71 | Gmd GDP-mannose 4,6-dehydratase from |
| | | | | | | | Aneurinibacillus thermoaerophilus |
| | | | AAD10232 | 5.00E−93 | 52 | 69 | putative GDP-D-mannose dehydratase |
| | | | | | | | from Anabaena sp. |
| | | | AAC44117 | 3.00E−89 | 50 | 68 | Gca GDP-D-mannose dehydratase involved in |
| | | | | | | | common antigen biosynthesis in |
| | | | | | | | Pseudomonas aeruginosa |
| | | | AAC38668 | 2.00E−88 | 49 | 67 | LpsA putative GDP-mannose-4,6-dehydratase |
| | | | | | | | predicted to be involved in S-layer lipopolysaccharide |
| | | | | | | | biosynthesis in Caulobacter crescentus |
| | | | AAF07199 | 3.00E−87 | 49 | 66 | Gmd1 GDP-D-mannose 4,6-dehydratase from |
| | | | | | | | Arabidopsis thaliana |
| 39 | 277 | resistance rRNA | AAG32067 | 2.00E−62 | 52 | 65 | AviRa rRNA methyltransferase involved in avilamycin |
| | | methyltransferase | | | | | A resistance in Streptomyces viridochromogenes |
| 40 | 159* | sugar epimerase/ | AAD35594 | 2.00E−31 | 43 | 63 | UDP-glucose 4-epimerase from Thermotoga maritima |
| | | ketoreductase | | | | | |
| | 49* | | C70562 | 2.00E−29 | 45 | 59 | robable dTDP-glucose 4-epimerase from |
| | | | | | | | Mycobacterium tuberculosis |
| | (partial) | | AAB98196 | 4.00E−28 | 43 | 61 | GalE UDP-glucose 4-epimerase from |
| | | | | | | | Methanococcus jannaschii |
| | | | CAA18814 | 2.00E−27 | 43 | 57 | putative sugar dehyratase from Mycobacterium leprae |
| 41 | 400 | flavoprotein | CAA51670 | 1.00E−108 | 55 | 68 | ORF3 flavoprotein in the daunorubicin biosynthetic |
| | | oxidoreductase | | | | | locus of Streptomyces griseus |
| | | | AAB63045 | 4.00E−56 | 39 | 47 | DnmZ putative flavoprotein required for biosynthesis |
| | | | | | | | of the daunorubicin precursor thymidine diphospho-L- |
| | | | | | | | daunosamine in Streptomyces peucetius |
| 42 | 373 | deoxyhexose | CAA11782 | 1.00E−157 | 73 | 82 | PCZA361.5 sugar biosynthesis gene in the |
| | | aminotransferase | | | | | chloroeremomycin biosynthetic locus of |
| | | | | | | | Amycolatopsis orientalis |
| | | | AAG13910 | 1.00E−151 | 70 | 83 | MegDII TDP-3-keto-6-deoxyhexose 3- |
| | | | | | | | aminotransaminase in the megalomicin |
| | | | | | | | biosynthetic locus of Micromonospora megalomicea |
| | | | AAF73462 | 1.00E−145 | 74 | 81 | AknZ putative aminotransferase in the aclacinomycin |
| | | | | | | | biosynthetic locus of Streptomyces galilaeus |
| | | | AAF01821 | 1.00E−143 | 73 | 81 | Snogl putative aminotransferase in the nogalamycin |
| | | | | | | | biosynthetic locus of Streptomyces nogalater |
| 43 | 416 | C-methyltransferase | CAA11777 | 1.00E−159 | 67 | 79 | PCZA361.22 sugar biosynthesis gene in the |
| | | | | | | | chloroeremomycin biosynthetic locus of |
| | | | | | | | Amycolatopsis orientalis |
| | | | AAC38444 | 1.00E−152 | 66 | 77 | DnrX daunorubicin/doxorubicin biosynthesis enzyme |
| | | | | | | | from Streptomyces peucetius |
| | | | CAB96549 | 2.00E−66 | 37 | 51 | MtmC D-mycarose 3-C-methyltransferase in the |
| | | | | | | | mithramycin biosynthetic locus of |
| | | | | | | | Streptomyces argillaceus |
| | | | AAG29803 | 7.00E−62 | 34 | 50 | CumW C-methyltransferase in the coumermycin A1 |
| | | | | | | | biosynthetic locus of Streptomyces rishiriensis |
| 44 | 207 | sugar epimerase | AAB63046 | 7.00E−68 | 63 | 75 | DnmU putative epimerase involved in the |
| | | | | | | | biosynthesis of daunorubicin precursor |
| | | | | | | | TDP-L-daunosamine in Streptomyces peucetius |
| | | | AAF70101 | 2.00E−64 | 60 | 73 | AknL dTDP-4-keto-6-deoxyhexose 3,5-epimerase in |
| | | | | | | | the aclacinomycin biosynthetic locus of |
| | | | | | | | Streptomyces galilaeus |
| | | | CAA11781 | 8.00E−64 | 58 | 72 | Protein similar to epimerase in the chloroeremomycin |
| | | | | | | | biosynthetic locus of Amycolatopsis orientalis |
| | | | CAA12011 | 1.00E−60 | 60 | 72 | SnogF 3,5-epimerase in the nogalamycin biosynthetic |
| | | | | | | | locus of Streptomyces nogalater |
| 45 | 343 | sugar ketoreductase | AAG13913 | 3.00E−86 | 54 | 64 | MegDV TDP-4-keto-6-deoxyhexose 4-ketoreductase |
| | | | | | | | in the megalomicin biosynthetic locus of |
| | | | | | | | Micromonospora megalomicea |
| | | | CAA11764 | 2.00E−84 | 51 | 71 | protein similar to dTDP-dehydrogenase in the |
| | | | | | | | chloroeremomycin biosynthetic locus of |
| | | | | | | | Amycolatopsis orientalis |
| | | | BAA84595 | 1.00E−79 | 53 | 63 | AveBlVdTDP-4-keto-6-deoxy-L-hexose 4-reductase in |
| | | | | | | | the avermectin biosynthetic locus of |
| | | | | | | | Streptomyces avermitilis |
| | | | AAB84071 | 3.00E−73 | 48 | 63 | EryBIV oxidoreductase involved in L-mycarose |
| | | | | | | | biosynthesis in the erythromycin biosynthetic |
| | | | | | | | locus of Saccharopolyspora erythraea |
| 46 | 306 | unknown | None | | | | |
| 47 | 518 | endoglucanase | AAA23084 | 2.00E−45 | 52 | 63 | endoglucanase from Cellulomonas fimi |
| | | | CAC16970 | 4.00E−41 | 35 | 47 | putative secreted endoglucanase from |
| | | | | | | | Streptomyces coeticolor |
| | | | AAA62211 | 5.00E−36 | 50 | 62 | beta-1,4-exocellulase precursor from |
| | | | | | | | Thermobifida fusca |
| 48 | 286 | transcriptional | CAB61919 | 2.00E−56 | 45 | 58 | putative lacl-family transcriptional regulator |
| | | regulator | | | | | in Streptomyces coelicolor |
| | | | CAA20609 | 8.00E−56 | 46 | 59 | putative lacl-family transcriptional regulator in |
| | | | | | | | Streptomyces coelicolor |
| | | | CAB65654 | 2.00E−28 | 28 | 48 | putative repressor of maltose transport |
| | | | | | | | genes in Alicyclobacillus |
| | | | | | | | acidocaldarius |
| | | | AAD51826 | 4.00E−28 | 34 | 49 | ThuR member of the Lacl-GalR family regulatory |
| | | | | | | | proteins in Sinorhizobium meliloti |
| 49 | 340 | glucokinase | CAB95296 | 4.00E−29 | 34 | 48 | probable sugar kinase from Streptomyces coelicolor |
| | | | CAB65576 | 6.00E−28 | 37 | 44 | putative transcriptional regulatory protein |
| | | | | | | | with similarity to glucokinase in |
| | | | | | | | Streptomyces coelicolor |
| | | | BAB05144 | 2.00E−27 | 31 | 47 | glucose kinase from Bacillus halodurans |
| | | | AAD36537 | 9.00E−26 | 29 | 45 | glucokinase from Thermotoga maritima |

The everninomicin backbone is composed of eight saccharide residues joined by glycosidic and orthoester linkages. Many of the proteins encoded by the everninomicin locus are likely to be involved in the biosynthesis of the sugar precursors and their subsequent joining and modification.

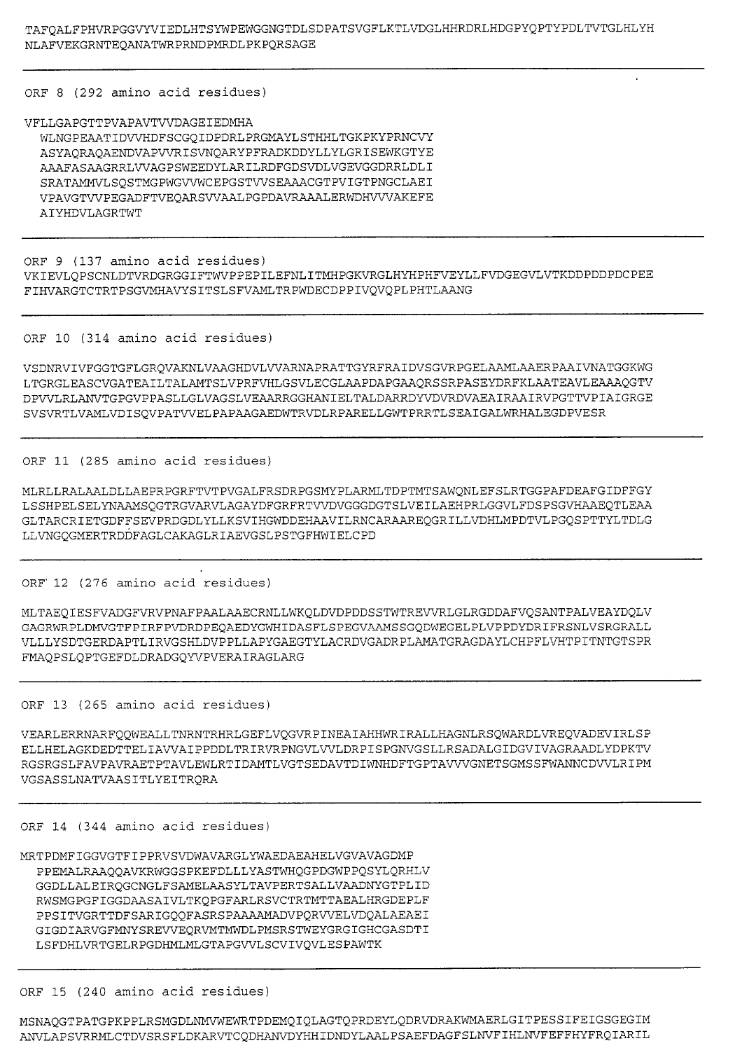

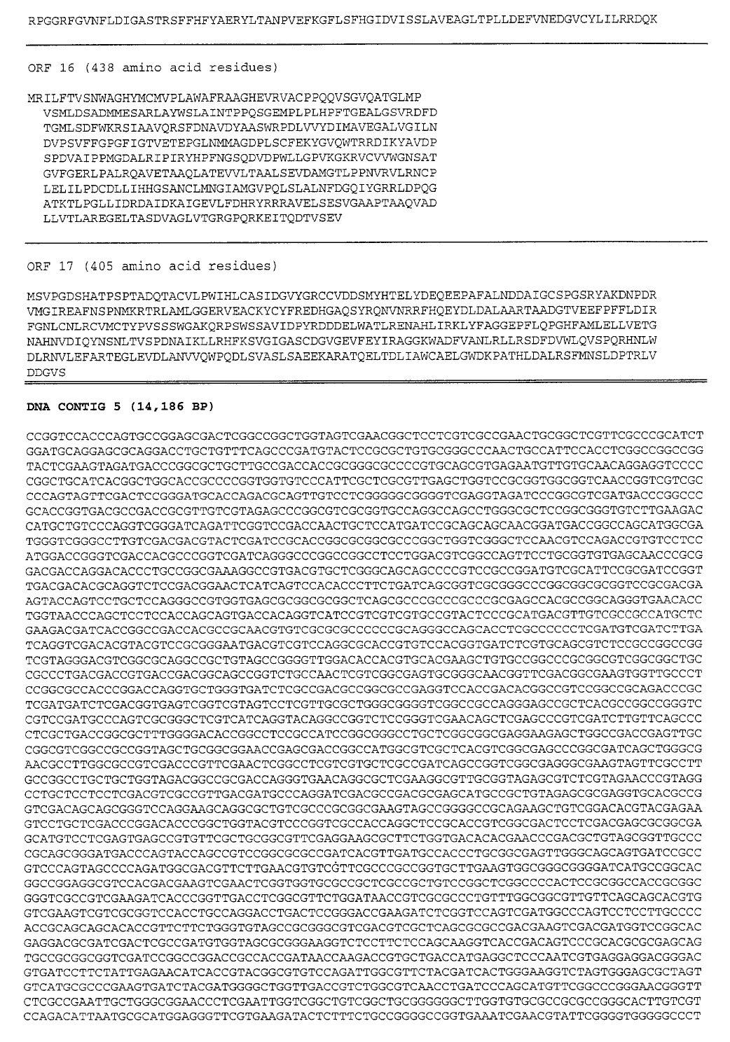

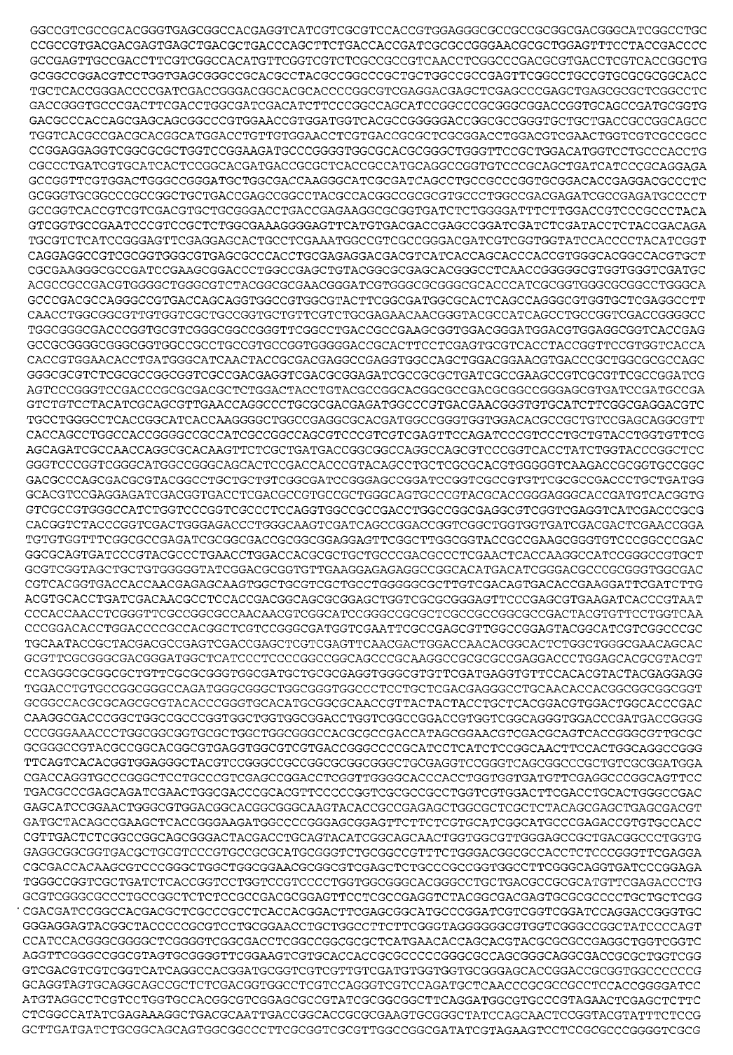

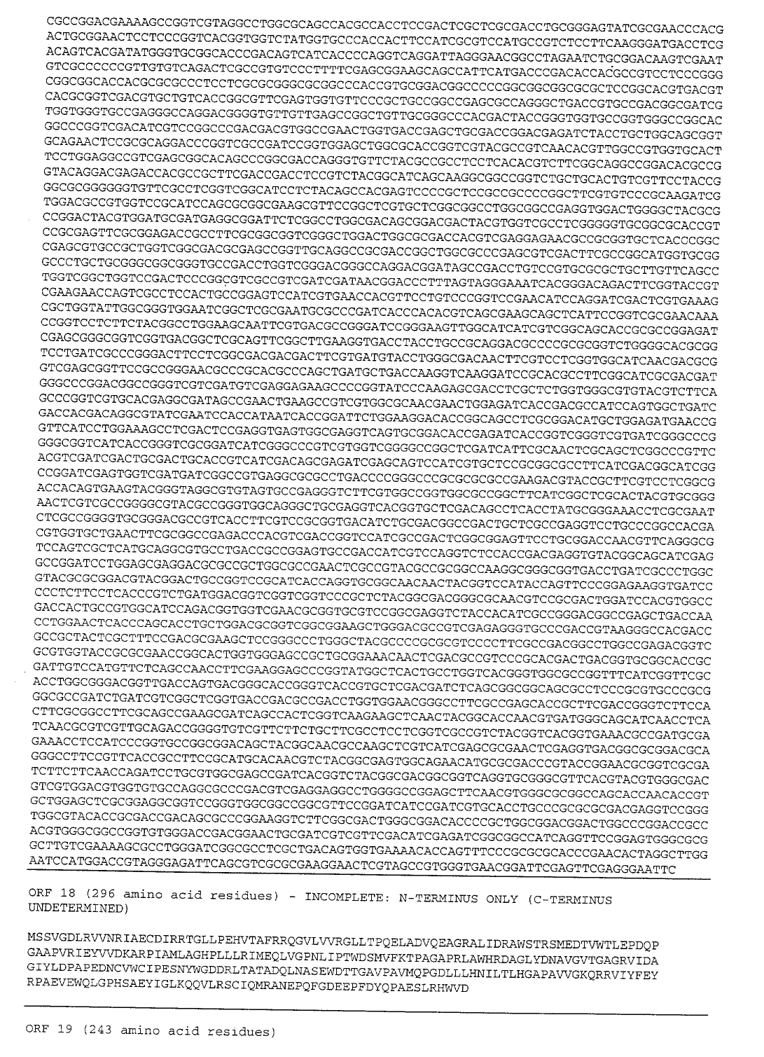

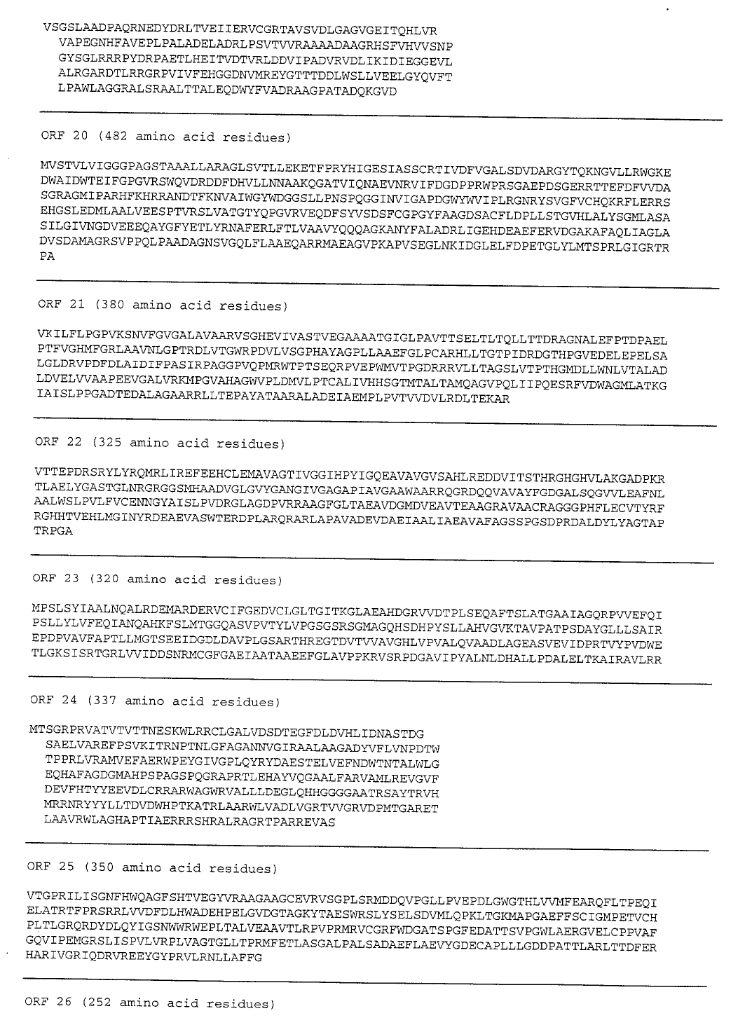

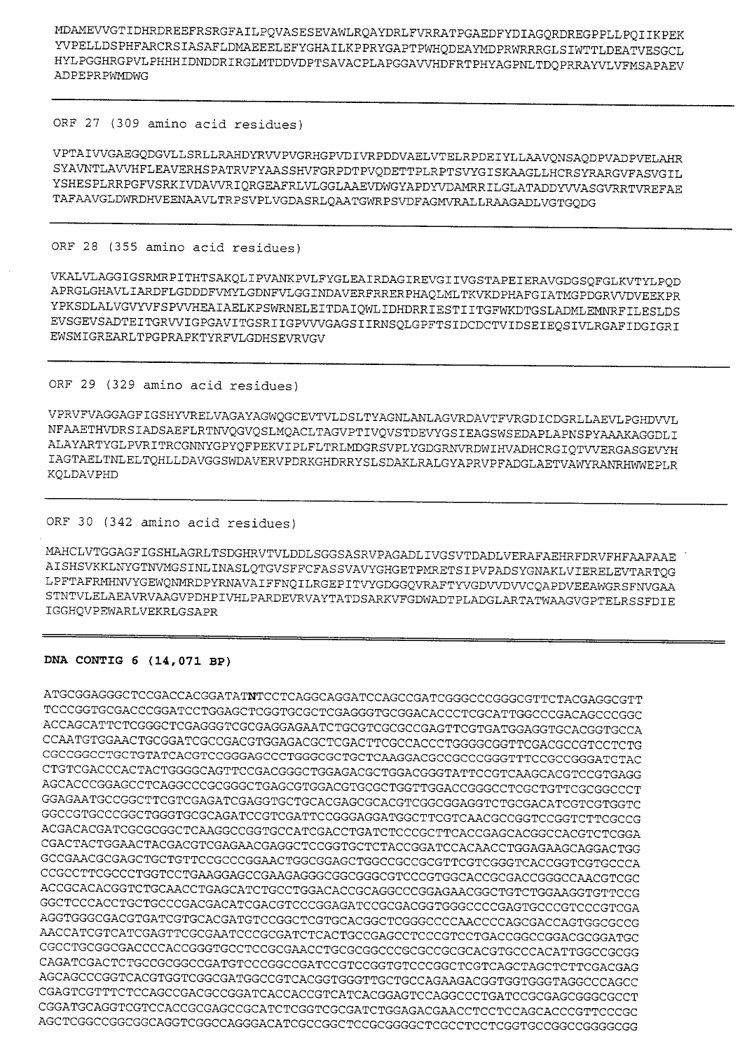

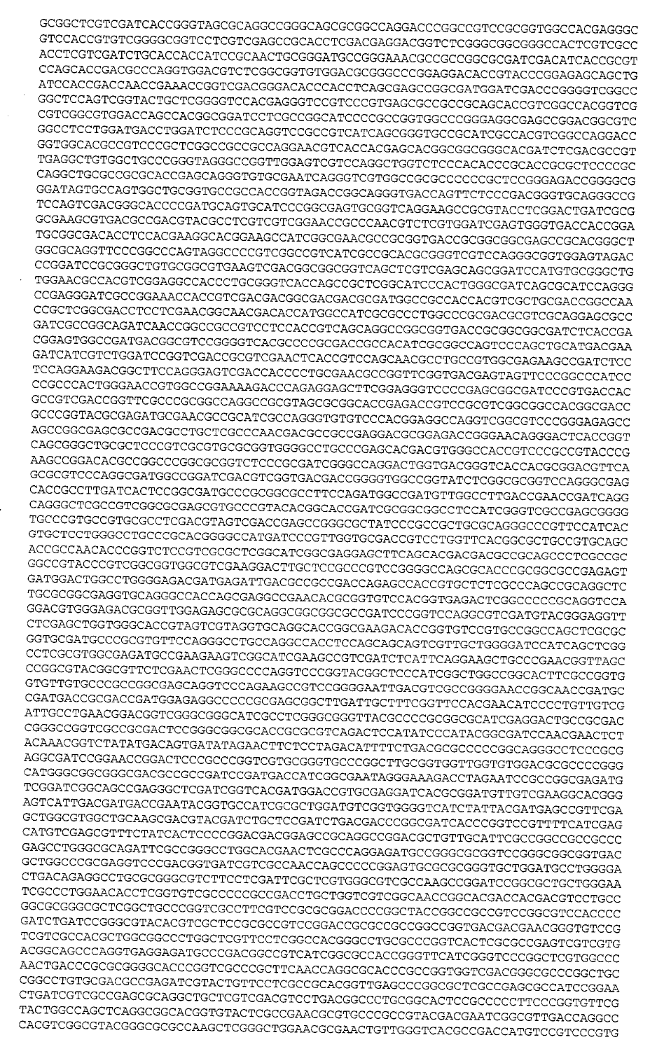

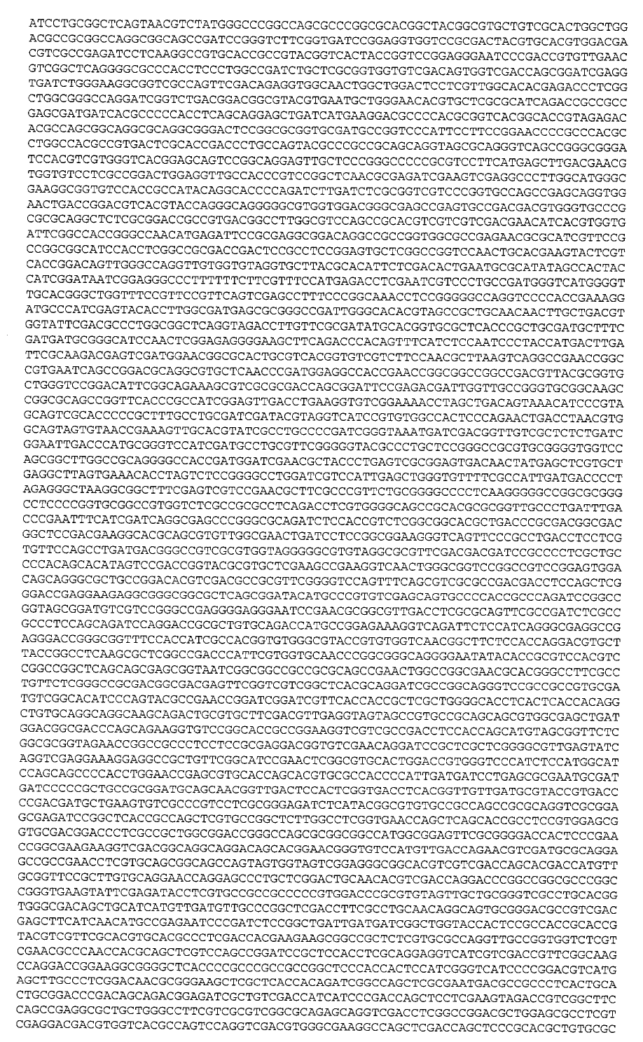

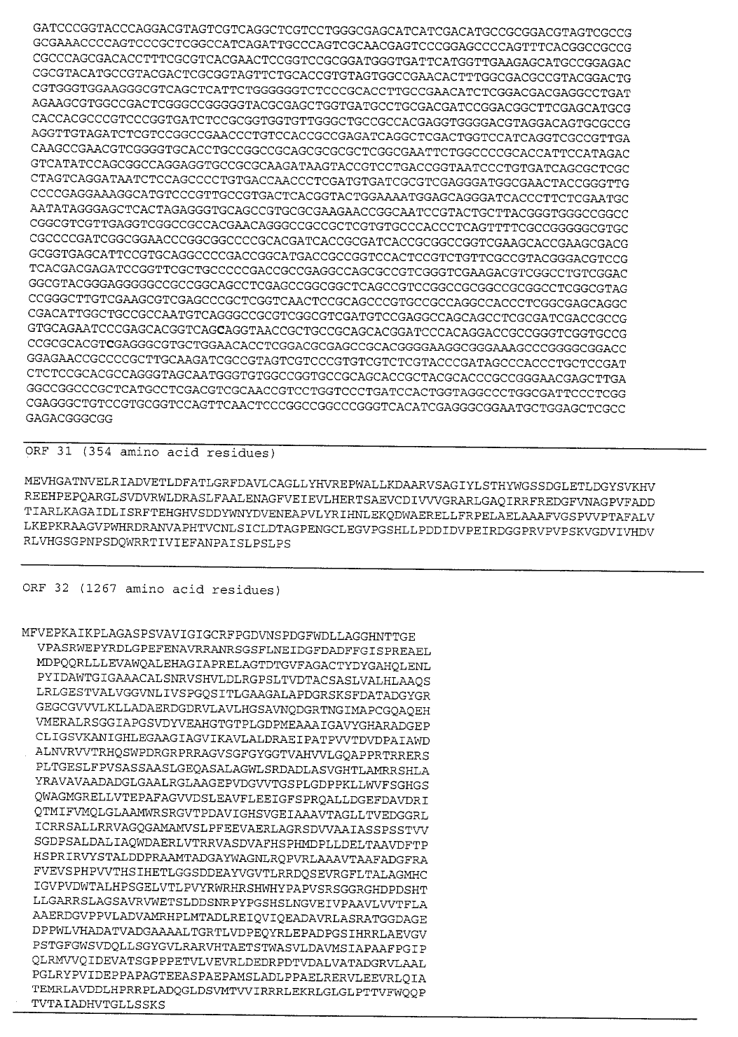

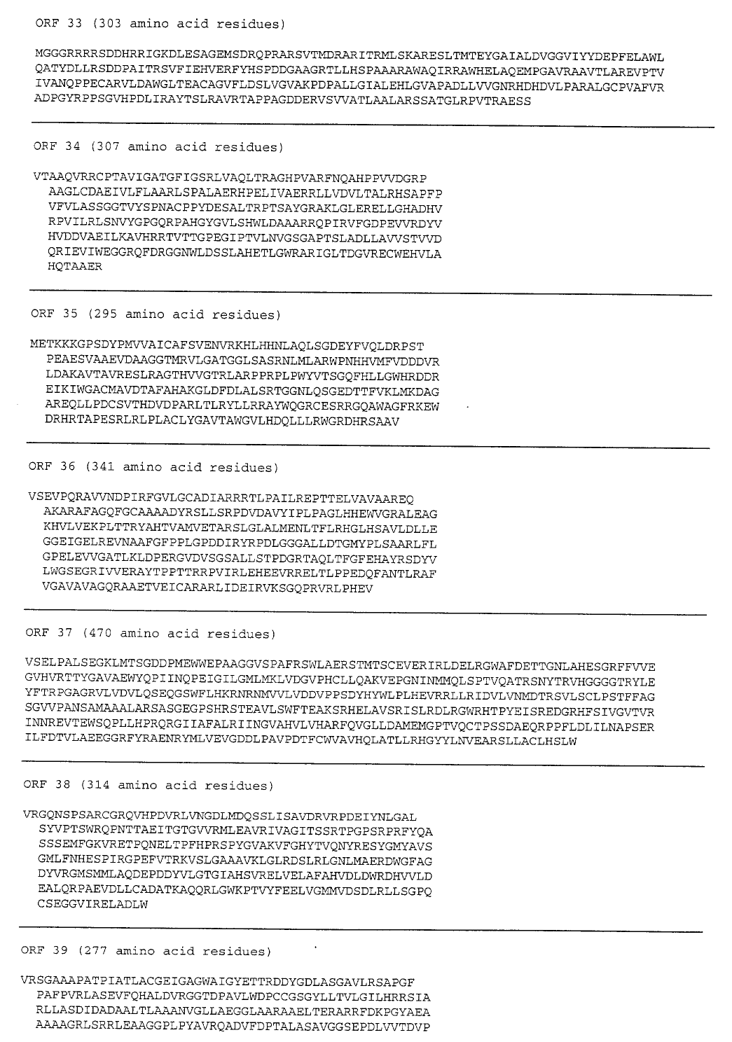

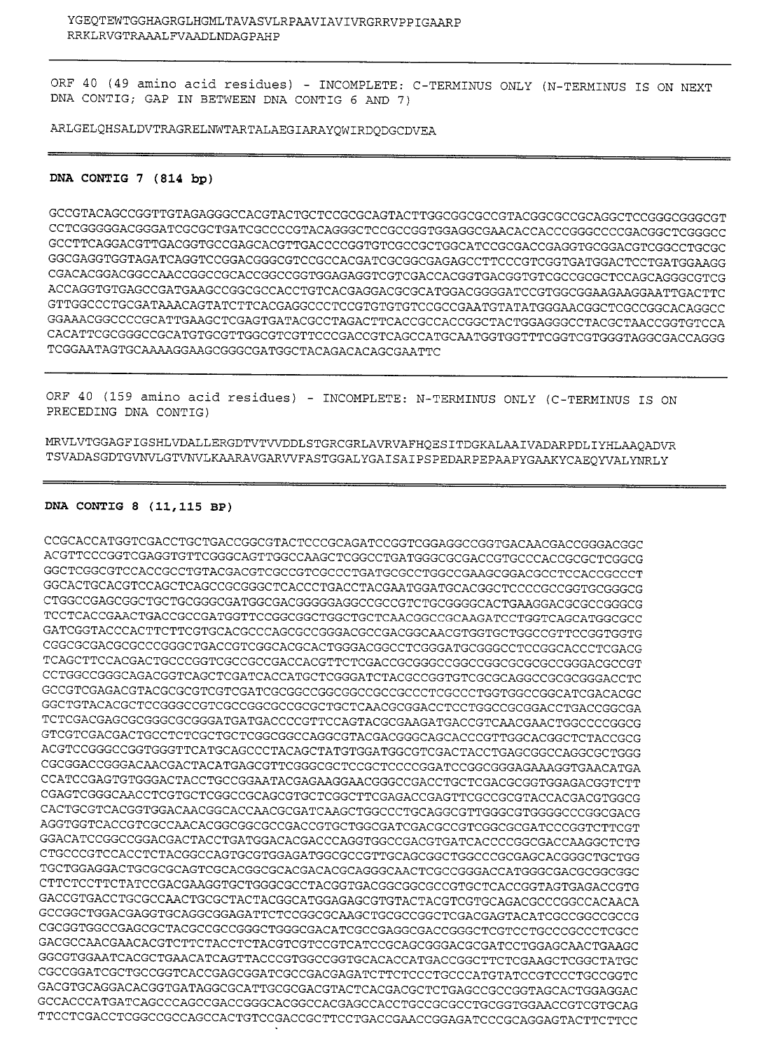

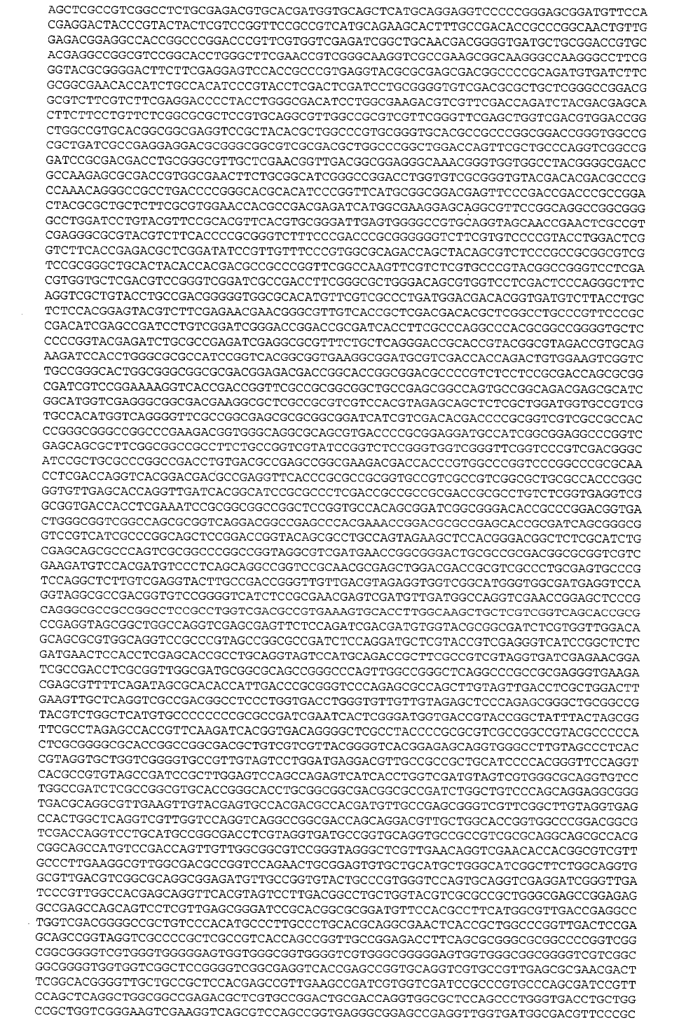

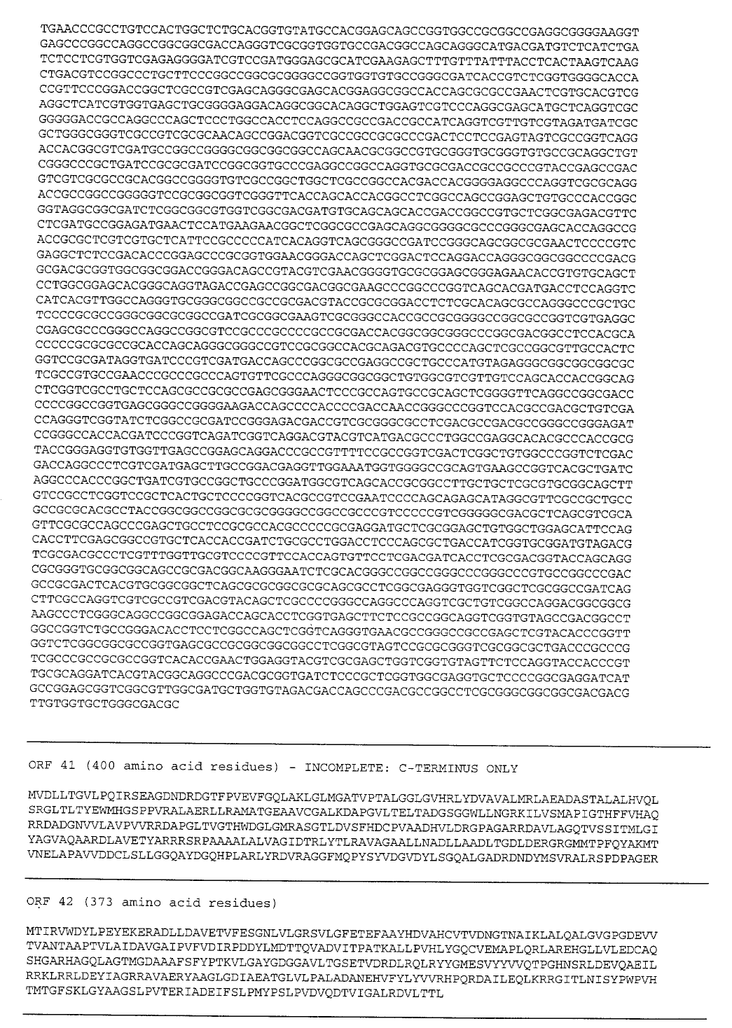

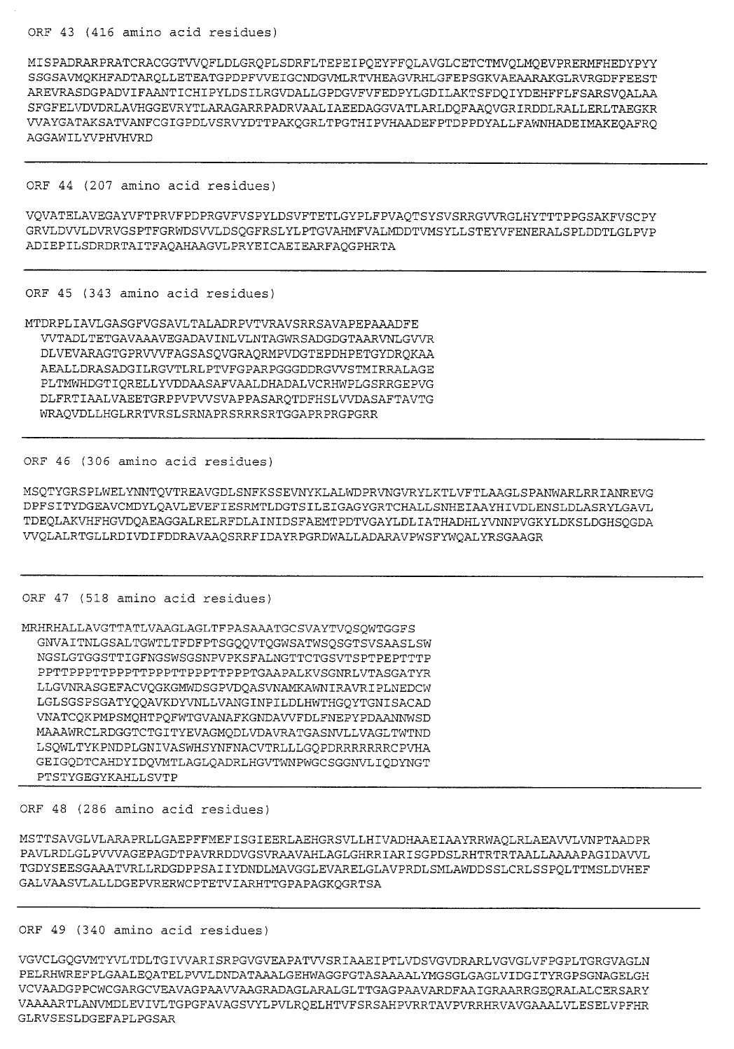

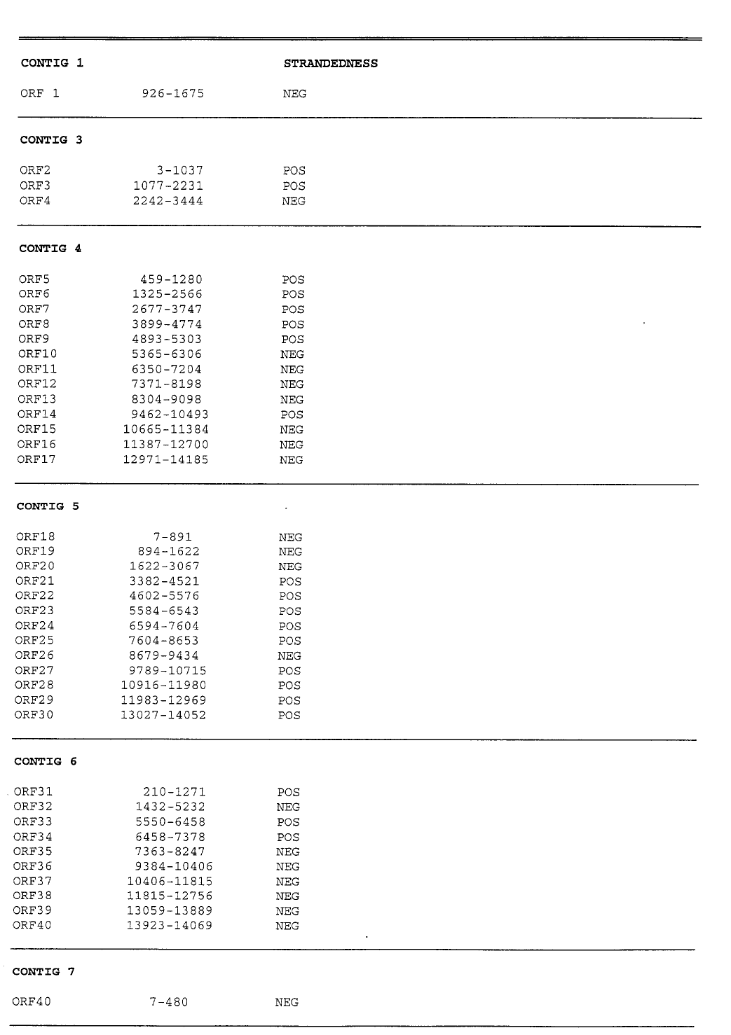

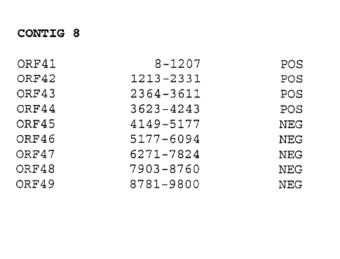

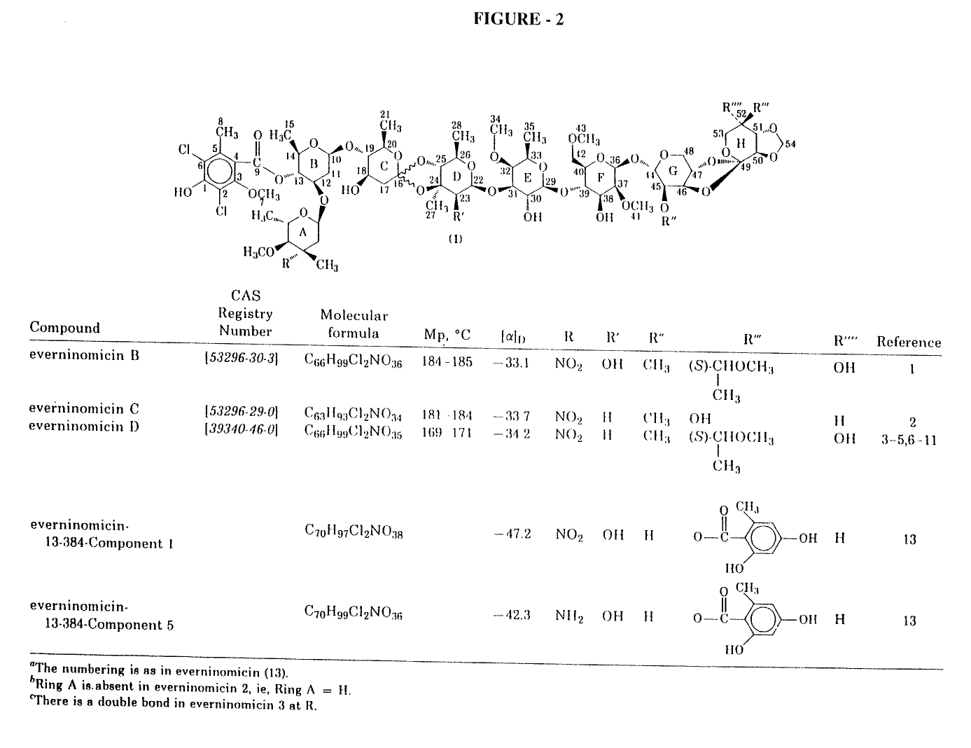

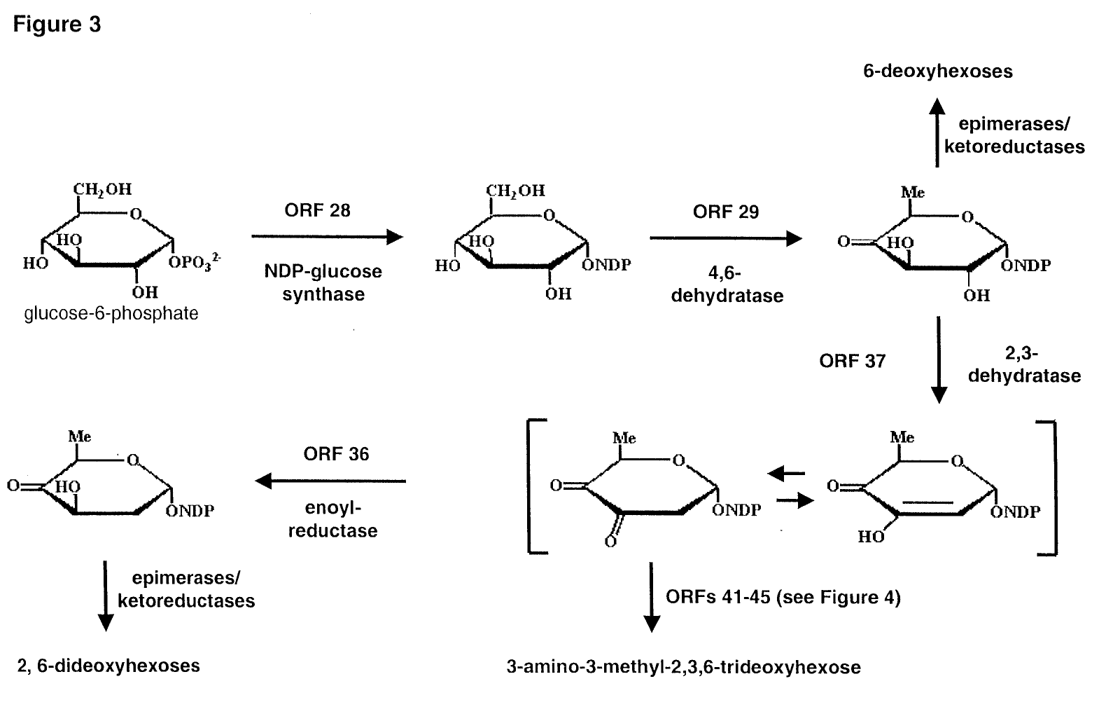

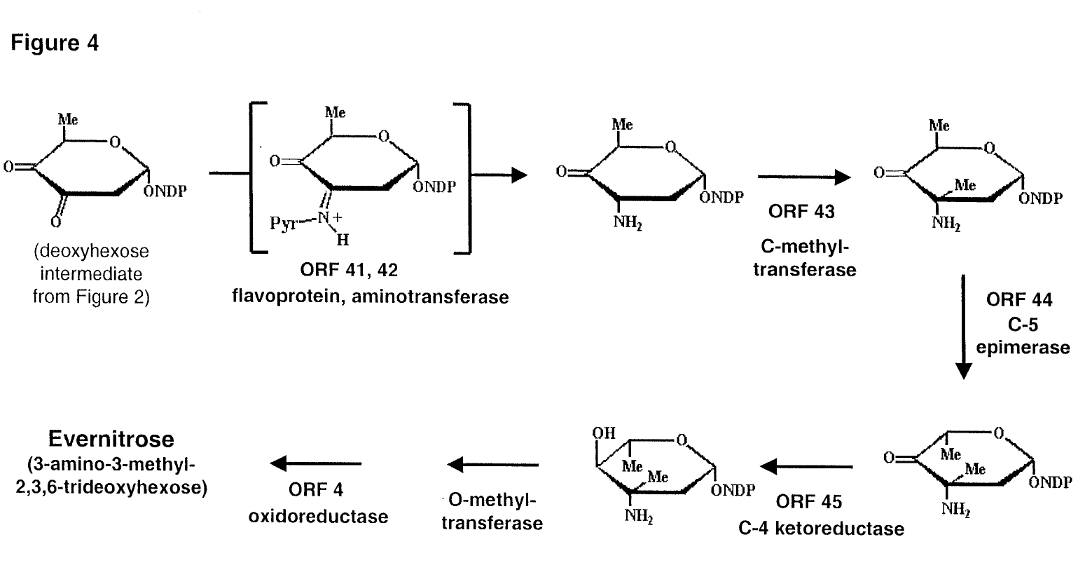

Five of the eight saccharide residues of everninomicin (residues A-E of FIG. 2) are deoxyhexoses and are likely to be derived from D-glucose-6-phosphate. Deoxyhexoses are common constituents of microbial secondary metabolites. The first two steps in the biosynthesis of many deoxysugars are the synthesis of dNDP-D-glucose and its conversion to dNDP-4-keto-6-deoxyglucose, catalyzed respectively by dNDP-glucose synthases and dNDP-glucose dehydratases (Liu and Thorson, 1994, Annu. Rev. Microbiol., Vol. 48, pp. 223-256). ORF 28 (SEQ ID NO 33) is similar to many bacterial dNDP-glucose synthases while ORF 29 (SEQ ID 34) is similar to many bacterial dNDP-glucose dehydratases. These two proteins are likely to be involved in generating 6-deoxyhexose precursors for incorporation into everninomicin. Sugar residues at positions A-C, and occasionally D, also lack C-2 hydroxyl groups (see FIG. 2). ORFs 36 and 37 (SEQ IS NOS 42 and 43) encode proteins that are similar to bacterial proteins known to be involved in C-2 deoxygenation and are therefore likely to be involved in the generation of 2,6-dideoxyhexose precursors. ORFs 10, 27, 30, 34, 38 and 40 (SEQ ID NOS 14, 32, 35, 40, 44, and 46) are similar to bacterial proteins that catalyze dehydration, epimerization and/or ketoreduction of deoxyhexose precursors and are likely to catalyze 4-ketoreduction to generate sugars with the appropriate C-4 stereochemistry for everninomicin biosynthesis. A biosynthetic scheme for the production of deoxyhexose precursors for everninomicin biosynthesis is shown in FIG. 3.

The everninomicins are distinguished from other orthosomycin antibiotics by the presence of a nitrogen-containing sugar residue (residue A of FIG. 2). ORFs 41-45 (SEQ ID NOS 50 to 54) constitute a cluster of ORFs with strong similarity to proteins involved in the biosynthesis of aminodeoxyhexoses. In particular, these ORFs are similar to proteins proposed to catalyze the synthesis of the 3-amino-3-methyl-2,3,6-trideoxyhexose residue of chloroeremomycin (van Wageningen et al., 1998, Chem. & Biol., Vol. 5, pp. 155-162) and proteins involved in the synthesis of the 3-amino-2,3,6-trideoxyhexose residue of daunorubicin (Olano et al., 1999, Chem. & Biol., Vol. 6, pp. 845-855). ORFs 41-45 (SEQ ID NOS 50 to 54) are therefore likely to catalyze the biosynthesis of a 3-amino-3-methyl-2,3,6-trideoxyhexose intermediate that would subsequently be modified by O-methyl transfer and amino group oxidation to yield the evernitrose nitrosugar residue. Two proteins (ORFs 1, 7; SEQ ID NOS 2 and 11) found in the everninomicin locus are similar to bacterial proteins that catalyze O-methyl transfer to deoxyhexoses groups of secondary metabolites and may catalyze O-methyl transfer in evernitrose biosynthesis. ORF 4 (SEQ ID NO 7) encodes an unusual oxidoreductase that shows similarity to bacterial blue-copper oxidoreductases involved in oxidizing nitrogen-containing compounds and as such provides a likely candidate for the amine oxidase required for the biosynthesis of evernitrose. A scheme for the biosynthesis of the nitrosugar evernitrose is shown in FIG. 4.

Five proteins (ORFs 8, 16, 21, 24 and 35; SEQ ID NOS 12, 20, 26, 29, and 41) are similar to bacterial glycosyltransferases and are therefore likely to catalyze the joining of saccharide precursors via glycosidic linkages to form the backbone oligosaccharide structure that is characteristic of the orthosomycins. Among the glycosyltransferases encoded by the everninomicin locus, one (ORF16; SEQ ID NO 20) shows the greatest similarity to enzymes known to catalyze the transfer of aminodeoxyhexose residues. This glycosyltransferase is therefore likely to catalyze the incorporation of the aminodeoxyhexose precursor that is subsequently converted to the nitrosugar evernitrose. The protein encoded by ORF 35 is the most unusual of the glycosyltransferases and is therefore likely to perform the unusual C-1 to C-1′ linkage that is characteristic of the orthosomycins.

The everninomicins may contain as many as 7 O-methyl groups (see FIG. 2). It is significant then that the everninomicin locus encodes seven proteins (ORFs 1, 3, 5, 7, 11, 15 and 19; SEQ ID NOS 2, 6, 9, 11, 19, and 24) that show similarity to O-methyltransferases. It is likely that each of these proteins catalyzes a specific O-methylation reaction during the course of everninomicin biosynthesis. ORFs 1 and 7 (SEQ ID NOS 2 and 11) are discussed above as possible enzymes responsible for methylating the C-4 hydroxyl group of the nitrosugar evernitrose. ORF 11 (SEQ ID NO 15) is discussed in more detail below and is likely to catalyze methylation of the phenolic hydroxyl group found on the dichloroisoeverninic acid moiety.

Four proteins encoded by the everninomicin locus (ORFs 12, 18, 26 and 31; SEQ ID NOS 16, 23, 32 and 37) are similar to oxidoreductases and are likely to catalyze the unusual oxidative modifications of the oligosaccharide backbone that are typical of the orthosomycins. In particular, three of these oxidoreductases (ORFs 18, 26 and 31; SEQ IS NOS 23, 31 and 37) show significant similarity to alpha-ketoglutarate-dependent dioxygenases and may therefore be involved in generating the three orthoester/diether linkages found in all orthosomycins (the orthoester linkages between sugar rings C-D and rings G-H, and the aliphatic methylene dioxy group appended to ring H, as shown in FIG. 2).

Two proteins in the everninomicin locus (ORFs 6, 43; SEQ ID NOS 10 and 52) are similar to C-methyltransferases that transfer methyl groups to deoxyhexose residues, thus accounting for the source of the two deoxyhexose C-methyl groups found in everninomicin (see FIG. 2). ORF 43 (SEQ ID NO 52) forms part of the aminodeoxyhexose gene cluster discussed earlier and is likely to be responsible for incorporating the C-3 methyl group of the evernitrose residue. ORF 6 (SEQ ID NO 10) is thus the likely source of the only remaining C-methyl group of everninomicin, that found on C-3 of the deoxyhexose residue D.

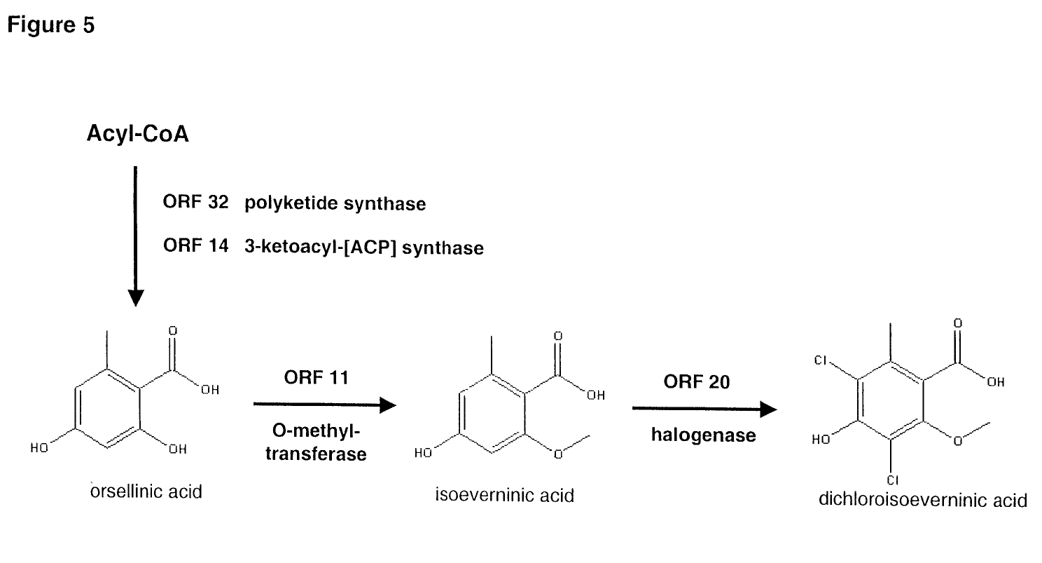

Four proteins encoded by the everninomicin locus (ORFs 11, 14, 20 and 32; SEQ ID NOS 15, 18, and 25) are likely to be involved in the biosynthesis of the dichloroisoeverninic moiety that is found in ester linkage to the sugar residue B of everninomicin (see FIG. 2). ORF 32 (SEQ ID NO 38) encodes a type I polyketide synthase that is similar to fungal 6-methylsalicylic acid synthases and to the AviM orsellinic acid synthase involved in avilamycin biosynthesis in Streptomyces viridochromogenes (Gaisser et al., 1997, J. Bacteriol., Vol. 179, pp. 6271-6278). ORF 32 (SEQ ID NO 38) is proposed to catalyze successive rounds of condensation of acyl-CoA precursors to form orsellinic acid, an aromatic precursor to isoeverninic acid. ORF 14 encodes a protein that is similar to 3-ketoacyl-[ACP]-synthases, including the DpsC protein in the daunorubicin biosynthetic locus of Streptomyces sp. strain C5. The DpsC protein has been proposed to interact with polyketide synthases and to confer specificity for the proper acyl-CoA starter unit (Rajgarhia et al., 1997, J. Bacteriol., Vol. 179, pp. 2690-2696). Similarly, the ORF 14 protein may interact with the ORF 32 (SEQ ID NO 38) polyketide synthase during the synthesis of the orsellinic acid precursor. ORF 11 (SEQ ID NO 15) encodes an O-methyltransferase that shows greatest similarity to bacterial proteins that transfer methyl groups to phenolic hydroxyls, and is therefore likely to catalyze the conversion of orsellinic acid to isoeverninic acid. ORF 20 (SEQ ID NO 25) encodes a protein that is similar to many bacterial non-heme halogenases, and is likely to catalyze the addition of 2 chlorine atoms to isoeverninic acid to form dichloroisoeverninic acid. A scheme for the biosynthesis of the dichioroisoeverninic acid moiety is shown in FIG. 5.

Three proteins encoded by the everninomicin locus (ORFs 22, 23 and 33; SEQ ID NOS 27, 28 and 39) are similar to enzymes involved in carbohydrate metabolism and may serve to generate short chain aliphatic alcohol precursors that are subsequently used to modify the variable positions on C-52 of residue H (see FIG. 2). ORFs 22 and 23 (SEQ ID NOS 27 and 28) are similar to subunits of the acetoin dehydrogenase component E1 involved in the catabolism of acetoin (3-hydroxy-2-butanone), while ORF 33 (SEQ ID NO 39) shows some similarity to bacterial phosphoglycolate phosphatases involved in glycolate (hydroxyacetic acid) metabolism.

Four proteins encoded by the everninomicin locus (ORFs 2, 13, 39 and 47; SEQ ID NOS 5, 17, 45 and 56)) are likely to be involved in conferring resistance to everninomicin and/or transporting everninomicin out of the producing bacterial cell. Everninomicin inhibits bacterial protein synthesis, and thus exerts its antibacterial effect, by binding to a specific site on the bacterial 50S ribosomal subunit (McNicholas et al., 2000, Antimicrob. Agents Chemother., Vol. 44, pp. 1121-1126). ORFs 13 and 39 (SEQ ID NOS 17 and 45) encode proteins that are similar to ribosomal RNA methyltransferases and are therefore likely to confer resistance to everninomicin (or its intermediates) by modifying the ribosomes of the producing microorganism. ORF 47 (SEQ ID NO 56) encodes a protein with similarity to a number of bacterial endoglucanases, enzymes that catalyze the hydrolysis of internal beta-1,4-glycosidic linkages. The ORF 47 (SEQ ID NO 56) enzyme may confer resistance to everninomicin or its intermediates by cleaving the beta-1,4-endoglycosidic linkage that is found in the oligosaccharide backbone of all orthosomycins. ORF 2 (SEQ ID NO 5) encodes a protein that is similar to integral membrane antiporters associated with antibiotic biosynthesis in other bacteria and is therefore likely to be involved in transport of everninomicin or its intermediates across the bacterial cell membrane.

Two proteins encoded by the everninomicin locus (ORFs 48, 49; SEQ ID NOS 57 and 58) are likely to be involved in regulating the expression of one or more of the genes in the locus. The orthosomycins are composed of repeating saccharide units and the biosynthesis of these molecules may be sensitive to the availability of saccharide precursors from primary cellular metabolism. ORF 48 (SEQ ID NO 57) encodes a protein that is similar to Lacl family transcriptional repressors that contain sugar binding sites and regulate transcription in response to the presence of small molecules such as saccharides. The ORF 49 (SEQ ID NO 58) protein is similar to glucose kinase and to ROK family transcriptional regulators that have glucose kinase homology. This protein may act as a sensor of hexose levels in the cell and interact with the ORF 48 (SEQ ID NO 57) transcriptional regulator in order to activate expression of one or more genes in the everninomicin locus in response to the availability of saccharide precursors.

Four proteins encoded by the everninomicin locus (ORFs 9, 17, 25 and 46; SEQ ID NOS 13, 21, 30 and 55) cannot be assigned a putative role in the biosynthesis of everninomicin. ORFs 17, 25 and 46 (SEQ ID NOS 21, 30 and 55) show no significant similarity to proteins in the GenBank database, while the ORF 9 (SEQ ID NO 13) protein shows weak similarity to putative nucleotide-binding proteins involved in sugar biosynthesis.

Polynucleotide and Amino Acid Sequences:

The term “isolated polynucleotide” is defined as a polynucleotide removed from the environment in which it naturally occurs. For example, a naturally-occurring DNA molecule present in the genome of a living bacteria is not isolated, but the same molecule separated from the remaining part of the bacterial genome, as a result of, e.g., a cloning event (amplification), is isolated. Typically, an isolated DNA molecule is free from its natural chromosomal context. Such isolated polynucleotides may be part of a vector or a composition and still be defined as isolated in that such a vector or composition is not part of the natural environment of such polynucleotide.

The polynucleotide of the invention is either RNA or DNA (cDNA, genomic DNA, or synthetic DNA), or modifications, variants, homologs or fragments thereof. The DNA is either double-stranded or single-stranded, and, if single-stranded, is either the coding strand or the non-coding (anti-sense) strand. Any one of the polynucleotide sequences of the invention as shown in FIG. 1 is (a) a coding sequence; (b) a ribonucleotide sequence derived from transcription of (a); (c) a coding sequence which uses the redundancy or degeneracy of the genetic code to encode the same polypeptides; or (d) a regulatory sequence. By “polypeptide” or “protein” is meant any chain of amino acids, regardless of length or post-translational modification (e.g., proteolytic processing or phosphorylation). Both terms are used interchangeably in the present application.

Consistent with this aspect of the invention, amino acid sequences are provided which are homologous to any one of the amino acid sequences of FIG. 1. As used herein, “homologous amino acid sequence” is any polypeptide which is encoded, in whole or in part, by a nucleic acid sequence which hybridizes at 25-35° C. below critical melting temperature (Tm), to any portion of the coding region nucleic acid sequences of FIG. 1. A homologous amino acid sequence is one that differs from an amino acid sequence shown in FIG. 1 by one or more conservative amino acid substitutions. Such a sequence also encompasses allelic variants (defined below) as well as sequences containing deletions or insertions which retain the functional characteristics of the polypeptide. Preferably, such a sequence is at least 75%, more preferably 80%, and most preferably 90% identical to any amino acid sequence shown in FIG. 1.

Homologous amino acid sequences include sequences that are identical or substantially identical to the amino acid sequences of FIG. 1. By “amino acid sequence substantially identical” is meant a sequence that is at least 90%, preferably 95%, more preferably 97%, and most preferably 99% identical to an amino acid sequence of reference and that preferably differs from the sequence of reference by a majority of conservative amino acid substitutions.

Conservative amino acid substitutions are substitutions among amino acids of the same class. These classes include, for example, amino acids having uncharged polar side chains, such as asparagine, glutamine, serine, threonine, and tyrosine; amino acids having basic side chains, such as lysine, arginine, and histidine; amino acids having acidic side chains, such as aspartic acid and glutamic acid; and amino acids having nonpolar side chains, such as glycine, alanine, valine, leucine, isoleucine, proline, phenylalanine, methionine, tryptophan, and cysteine.

Homology is measured using sequence analysis software such as Sequence Analysis Software Package of the Genetics Computer Group, University of Wisconsin Biotechnology Center, 1710 University Avenue, Madison, Wis. 53705. Amino acid sequences are aligned to maximize identity. Gaps may be artificially introduced into the sequence to attain proper alignment. Once the optimal alignment has been set up, the degree of homology is established by recording all of the positions in which the amino acids of both sequences are identical, relative to the total number of positions.

Homologous polynucleotide sequences are defined in a similar way. Preferably, a homologous sequence is one that is at least 45%, more preferably 60%, and most preferably 85% identical to any one of the coding sequences of FIG. 1.

Consistent with this aspect of the invention, polypeptides having a sequence homologous to any one of the amino acid sequences of FIG. 1 include naturally-occurring allelic variants, as well as mutants or any other non-naturally occurring variants that retain the inherent characteristics of any polypeptide of FIG. 1.

As is known in the art, an allelic variant is an alternate form of a polypeptide that is characterized as having a substitution, deletion, or addition of one or more amino acids that does not alter the biological function of the polypeptide. By “biological function” is meant the function of the polypeptide in the cells in which it naturally occurs. A polypeptide can have more than one biological function.

Also consistent with this aspect of the invention is a substantially purified polypeptide or polypeptide derivative having an amino acid sequence encoded by a polynucleotide of the invention. A “substantially purified polypeptide” as used herein is defined as a polypeptide that is separated from the environment in which it naturally occurs and/or that is free of the majority of the polypeptides that are present in the environment in which it was synthesized. For example, a substantially purified polypeptide is free from cellular polypeptides. Those skilled in the art would readily understand that the polypeptides of the invention may be purified from a natural source, i.e., a bacterial cell of the order Actinomycetales, or produced by recombinant means.

The nucleic acids of ORF 1 to 49 can be isolated, optionally modified and inserted into a host cell to create and/or modify a metabolic (biosynthetic) and thereby enable that host cell to synthesize and/or modify various metabolites.

Alternatively, the everninomicin gene cluster can be expressed in the host cell and the encoded everninomicin polypeptides recovered for use as chemical reagents, e.g. in the ex vivo synthesis and/or chemical modification of various metabolites. Either application typically entails insertion of one or more nucleic acids encoding one or more isolated and/or modified everninomicin open reading frames in a metabolic/biosynthetic pathway (in which case the synthetic product of the pathway is typically recovered) or the everninomicin polypeptides themselves are recovered. The nucleic acid(s) are typically in an expression vector, a construct containing control elements suitable to direct expression of the everninomicin polypeptides. The expressed everninomicin polypeptides in the host cell then act as components of a metabolic/biosynthetic pathway (in which case the synthetic product of the pathway is typically recovered) or the everninomicin polypeptides themselves are recovered. Using the sequence information provided herein, cloning and expression of everninomicin nucleic acids can be accomplished using routine and well-known methods.

The ORFs (SEQ ID NOS: 2, 5 to 7, 9 to 21, 23 to 35, 37 to 46, 48, and 50 to 58) can be used to synthesize everninomicin antibiotics and/or analogues thereof. Alternatively, various components of the everninomicin gene cluster can be used to synthesize and/or chemically modify a wide variety of biomolecules/metabolites.

Polynucleotides encoding homologous polypeptides or allelic variants are retrieved by polymerase chain reaction (PCR) amplification of genomic bacterial DNA extracted by conventional methods. This involves the use of synthetic oligonucleotide primers matching upstream and downstream of the 5′ and 3′ ends of the encoding domain. Suitable primers are designed according to the nucleotide sequence information provided in FIG. 1. The procedure is as follows: a primer is selected which consists of 10 to 40, preferably 15 to 25 nucleotides. It is advantageous to select primers containing C and G nucleotides in a proportion sufficient to ensure efficient hybridization; i.e., an amount of C and G nucleotides of at least 40%, preferably 50% of the total nucleotide content. A standard PCR reaction contains typically 0.5 to 5 Units of Taq DNA polymerase per 100 μL, 20 to 200 μM deoxynucleotide each, preferably at equivalent concentrations, 0.5 to 2.5 mM magnesium over the total deoxynucleotide concentration, 10⁵ to 10⁶ target molecules, and about 20 pmol of each primer. About 25 to 50 PCR cycles are performed, with an annealing temperature 15° C. to 5° C. below the true Tm of the primers. A more stringent annealing temperature improves discrimination against incorrectly annealed primers and reduces incorportion of incorrect nucleotides at the 3′ end of primers. A denaturation temperature of 95° C. to 97° C. is typical, although higher temperatures may be appropriate for denaturation of G+C-rich targets. The number of cycles performed depends on the starting concentration of target molecules, though typically more than 40 cycles is not recommended as non-specific background products tend to accumulate.

An alternative method for retrieving polynucleotides encoding homologous polypeptides or allelic variants is by hybridization screening of a DNA or RNA library. Hybridization procedures are well-known in the art and are described in Ausubel et al., (Ausubel et al., Current Protocols in Molecular Biology, John Wiley & Sons Inc., 1994), Silhavy et al. (Silhavy et al. Experiments with Gene Fusions, Cold Spring Harbor Laboratory Press, 1984), and Davis et al. (Davis et al. A Manual for Genetic Engineering: Advanced Bacterial Genetics, Cold Spring Harbor Laboratory Press, 1980). Important parameters for optimizing hybridization conditions are reflected in a formula used to obtain the critical melting temperature above which two complementary DNA strands separate from each other (Casey & Davidson, Nucl. Acid Res. (1977) 4:1539). For polynucleotides of about 600 nucleotides or larger, this formula is as follows: Tm=81.5+0.5×(% G+C)+1.6 log (positive ion concentration)−0.6×(% formamide). Under appropriate stringency conditions, hybridization temperature (Th) is approximately 20 to 40° C., 20 to 25° C., or, preferably 30 to 40° C. below the calculated Tm. Those skilled in the art will understand that optimal temperature and salt conditions can be readily determined.

For the polynucleotides of the invention, stringent conditions are achieved for both pre-hybridizing and hybridizing incubations (i) within 4-16 hours at 42° C., in 6×SSC containing 50% formamide, or (ii) within 4-16 hours at 65° C. in an aqueous 6×SSC solution (1 M NaCl, 0.1M sodium citrate (pH 7.0)).

The native everninomicin gene cluster ORFs can be re-ordered, modified and combined with other biosynthetic units to produce a wide variety of molecules. Large chemical libraries can be produced and screened for a desired activity.

Useful homologs and fragments thereof that do not occur naturally are designed using known methods for identifying regions of a polypeptide that are likely to tolerate amino acid sequence changes and/or deletions. As an example, homologous polypeptides from different species are compared; conserved sequences are identified. The more divergent sequences are the most likely to tolerate sequence changes. Homology among sequences may be analyzed using the BLAST homology searching algorithm of Altschul et al., Nucleic Acids Res. 25:3389-3402 (1997).

Alternatively, identification of homologous polypeptides or polypeptide derivatives encoded by polynucleotides of the invention which have activity in the everninomicin biosynthetic pathway may be achieved by screening for cross-reactivity with an antibody raised against the polypeptide of reference having an amino acid sequence of FIG. 1. The procedure is as follows: an antibody is raised against a purified reference polypeptide, a fusion polypeptide (for example, an expression product of MBP, GST, or His-tag systems), or a synthetic peptide derived from the reference polypeptide. Where an antibody is raised against a fusion polypeptide, two different fusion systems are employed. Specific antigenicity can be determined according to a number of methods, including Western blot (Towbin et al., Proc. Natl. Acad. Sci. USA (1979) 76:4350), dot blot, and ELISA, as described below.

In a Western blot assay, the product to be screened, either as a purified preparation or a total E. coli extract, is submitted to SDS-Page electrophoresis as described by Laemmli (Nature (1970) 227:680). After transfer to a nitrocellulose membrane, the material is further incubated with the antibody diluted in the range of dilutions from about 1:5 to about 1:5000, preferably from about 1:100 to about 1:500. Specific antigenicity is shown once a band corresponding to the product exhibits reactivity at any of the dilutions in the above range.

In an ELISA assay, the product to be screened is preferably used as the coating antigen. A purified preparation is preferred, although a whole cell extract can also be used. Briefly, about 100 μl of a preparation at about 10 μg protein/ml are distributed into wells of a 96-well polycarbonate ELISA plate. The plate is incubated for 2 hours at 37° C. then overnight at 4° C. The plate is washed with phosphate buffer saline (PBS) containing 0.05% Tween 20 (PBS/Tween buffer). The wells are saturated with 250 μl PBS containing 1% bovine serum albumin (BSA) to prevent non-specific antibody binding. After 1 hour incubation at 37° C., the plate is washed with PBS/Tween buffer. The antibody is serially diluted in PBS/Tween buffer containing 0.5% BSA. 100 μl of dilutions are added per well. The plate is incubated for 90 minutes at 37° C., washed and evaluated according to standard procedures. For example, a goat anti-rabbit peroxidase conjugate is added to the wells when specific antibodies were raised in rabbits. Incubation is carried out for 90 minutes at 37° C. and the plate is washed. The reaction is developed with the appropriate substrate and the reaction is measured by colorimetry (absorbance measured spectrophotometrically). Under the above experimental conditions, a positive reaction is shown by O.D. values greater than a non immune control serum.

In a dot blot assay, a purified product is preferred, although a whole cell extract can also be used. Briefly, a solution of the product at about 100 μg/ml is serially two-fold diluted in 50 mM Tris-HCl (pH 7.5). 100 μl of each dilution are applied to a nitrocellulose membrane 0.45 μm set in a 96-well dot blot apparatus (Biorad). The buffer is removed by applying vacuum to the system. Wells are washed by addition of 50 mM Tris-HCl (pH 7.5) and the membrane is air-dried. The membrane is saturated in blocking buffer (50 mM Tris-HCl (pH 7.5) 0.15 M NaCl, 10 g/L skim milk) and incubated with an antibody dilution from about 1:50 to about 1:5000, preferably about 1:500. The reaction is revealed according to standard procedures. For example, a goat anti-rabbit peroxidase conjugate is added to the wells when rabbit antibodies are used. Incubation is carried out 90 minutes at 37° C. and the blot is washed. The reaction is developed with the appropriate substrate and stopped. The reaction is measured visually by the appearance of a colored spot, e.g., by colorimetry. Under the above experimental conditions, a positive reaction is shown once a colored spot is associated with a dilution of at least about 1:5, preferably of at least about 1:500.

Another aspect of the invention provides a process for purifying a polypeptide or polypeptide derivative of the invention by affinity chromatography using as a ligand either an antibody or an orthosomycin-related compound which binds to the polypeptide. The antibody is either polyclonal or monoclonal. Purified IgGs are prepared from an antiserum using standard methods (see, e.g., Coligan et al., Current Protocols in Immunology (1994) John Wiley & Sons, Inc., New York, N.Y.). Conventional chromatography supports are described in, e.g., Antibodies: A Laboratory Manual, D. Lane, E. Harlow, Eds. (1988).

Consistent with this aspect of the invention, polypeptide derivatives are provided that are partial sequences of the amino acid sequences of FIG. 1, partial sequences of polypeptide sequences homologous to the amino acid sequences of FIG. 1, polypeptides derived from full-length polypeptides by internal deletion, and fusion proteins.

Polynucleotides of 30 to 600 nucleotides encoding partial sequences of sequences homologous to nucleotide sequences of FIG. 1 are retrieved by PCR amplification using the parameters outlined above and using primers matching the sequences upstream and downstream of the 5′ and 3′ ends of the fragment to be amplified. The template polynucleotide for such amplification is either the full length polynucleotide homologous to a polynucleotide sequence of FIG. 1, or a polynucleotide contained in a mixture of polynucleotides such as a DNA or RNA library. As an alternative method for retrieving the partial sequences, screening hybridization is carried out under conditions described above and using the formula for calculating Tm. Where fragments of 30 to 600 nucleotides are to be retrieved, the calculated Tm is corrected by subtracting (600/polynucleotide size in base pairs) and the stringency conditions are defined by a hybridization temperature that is 5 to 10° C. below Tm. Where oligonucleotides shorter than 20-30 bases are to be obtained, the formula for calculating the Tm is as follows: Tm=4×(G+C)+2×(A+T). For example, an 18 nucleotide fragment of 50% G+C would have an approximate Tm of 54° C. Short peptides that are fragments of the polypeptide sequences of FIG. 1 or their homologous sequences, are obtained directly by chemical synthesis (E. Gross and H. J. Meinhofer, 4 The Peptides: Analysis, Synthesis, Biology; Modern Techniques of Peptide Synthesis, John Wiley & Sons (1981), and M. Bodanzki, Principles of Peptide Synthesis, Springer-Verlag (1984)).

Polynucleotides encoding polypeptide fragments and polypeptides having large internal deletions are constructed using standard methods (Ausubel et al., Current Protocols in Molecular Biology, John Wiley & Sons Inc., 1994). Such methods include standard PCR, inverse PCR, restriction enzyme treatment of cloned DNA molecules, or the method of Kunkel et al. (Kunkel et al Proc. Natl. Acad. Sci. USA (1985) 82:448). Components for these methods and instructions for their use are readily available from various commercial sources such as Stratagene. Once the deletion mutants have been constructed, they are tested for their ability to improve production of everninomicin or generate novel analogues of the antibiotic or natural products of the orthosomycin class as described above.

As used herein, a fusion polypeptide is one that contains a polypeptide or a polypeptide derivative of the invention fused at the N- or C-terminal end to any other polypeptide (hereinafter referred to as a peptide tail). A simple way to obtain such a fusion polypeptide is by translation of an in-frame fusion of the polynucleotide sequences, i.e., a hybrid gene. The hybrid gene encoding the fusion polypeptide is inserted into an expression vector which is used to transform or transfect a host cell. Alternatively, the polynucleotide sequence encoding the polypeptide or polypeptide derivative is inserted into an expression vector in which the polynucleotide encoding the peptide tail is already present. Such vectors and instructions for their use are commercially available, e.g. the pMal-c2 or pMal-p2 system from New England Biolabs, in which the peptide tail is a maltose binding protein, the glutathione-S-transferase system of Pharmacia, or the His-Tag system available from Novagen. These and other expression systems provide convenient means for further purification of polypeptides and derivatives of the invention.

Vectors, Transformed Cells, Primers and Probes:

A polynucleotide molecule according to the invention, including RNA, DNA, or modifications or combinations thereof, have various applications. A DNA molecule is used, for example, for producing a polypeptide of the invention in a recombinant host system. Another aspect of the invention encompasses (a) an expression cassette containing a DNA molecule of the invention placed under the control of the elements required for expression, in particular under the control of an appropriate promoter; (b) an expression vector containing an expression cassette of the invention; (c) a prokaryotic cell transformed with an expression cassette and/or vector of the invention, as well as (d) a process for producing a polypeptide or polypeptide derivative encoded by a polynucleotide of the invention, which involves culturing a prokaryotic cell transformed with an expression cassette and/or vector of the invention under conditions that allow expression of the DNA molecule of the invention, and recovering the encoded polypeptide or polypeptide derivative from the culture.

A recombinant expression system is selected from prokaryotic hosts. Bacterial cells are available from a number of different sources including commercial sources to those skilled in the art, e.g., the American Type Culture Collection (ATCC; Rockville, Md.). Commercial sources of cells used for recombinant protein expression also provide instructions for usage of the cells.

The choice of the expression system depends on the features desired for the expressed polypeptide. For example, it may be useful to produce a polypeptide of the invention in a particular lipidated form or any other form.

One skilled in the art would readily understand that not all vectors and expression control sequences and hosts would be expected to express equally well the polynucleotides of this invention. With the guidelines described below, however, a selection of vectors, expression control sequences and hosts may be made without undue experimentation and without departing from the scope of this invention.

In selecting a vector, the host must be chosen that is compatible with the vector which is to exist and possibly replicate in it. Considerations are made with respect to the vector copy number, the ability to control the copy number and expression of other proteins such as antibiotic resistance. In selecting an expression control sequence, a number of variables are considered. Among the important variables are the relative strength of the sequence (e.g. the ability to drive expression under various conditions), the ability to control the sequence's function and compatibility between the polynucleotide to be expressed and the control sequence (e.g. secondary structures are considered to avoid hairpin structures which prevent efficient transcription). In selecting the host, unicellular hosts are selected which are compatible with the selected vector, tolerant of any possible toxic effects of the expressed product, able to secrete the expressed product efficiently if such is desired, able to express the product in the desired conformation, easily scaled up, and having regard to ease of purification of the final product, which may be the expressed polypeptide or the natural product, e.g. an antibiotic, which is a product of the biosynthetic pathway of which the expressed polypeptide is a part.

The choice of the expression cassette depends on the host system selected as well as the features desired for the expressed polypeptide or natural product. Typically, an expression cassette includes a promoter that is functional in the selected host system and can be constitutive or inducible; a ribosome binding site; a start codon (ATG) if necessary; optionally a region encoding a leader peptide; a DNA molecule of the invention; a stop codon; and optionally a 3′ terminal region (translation and/or transcription terminator). The leader peptide encoding region is adjacent to the polynucleotide of the invention and placed in proper reading frame. The leader peptide-encoding region, if present, is homologous or heterologous to the DNA molecule encoding the mature polypeptide and is compatible with the secretion apparatus of the host used for expression. The open reading frame constituted by the DNA molecule of the invention, solely or together with the leader peptide, is placed under the control of the promoter so that transcription and translation occur in the host system. Promoters and leader peptide encoding regions are widely known and available to those skilled in the art.

The expression cassette is typically part of an expression vector, which is selected for its ability to replicate in the chosen expression system. Expression vectors (e.g., plasmids and cosmids) are widely known and are readily available to those skilled in the art. For bacterial vectors, the polynucleotide of the invention is inserted into the bacterial genome or remains in a free state as part of a plasmid. Methods for transforming host cells with expression vectors are well-known in the art.

The sequence information provided in the present application enables the design of specific nucleotide probes and primers that are used for identifying and isolating putative orthosomycin-producing microorganisms. Accordingly, an aspect of the invention provides a nucleotide probe or primer having a sequence found in or derived by degeneracy of the genetic code from a sequence shown in FIG. 1.

The term “probe” as used in the present application refers to DNA (preferably single stranded) or RNA molecules (or modifications or combinations thereof) that hybridize under the stringent conditions, as defined above, to nucleic acid molecules of FIG. 1 or to sequences homologous to those of FIG. 1, or to their complementary or anti-sense sequences. Generally, probes are significantly shorter than full-length sequences. Such probes contain from about 5 to about 100, preferably from about 10 to about 80, nucleotides. In particular, probes have sequences that are at least 75%, preferably at least 85%, more preferably 95% homologous to a portion of a sequence disclosed in FIG. 1 or that are complementary to such sequences. Probes may contain modified bases such as inosine, methyl-5-deoxycytidine, deoxyuridine, dimethylamino-5-deoxyuridine, or diamino-2, 6-purine. Sugar or phosphate residues may also be modified or substituted. For example, a deoxyribose residue may be replaced by a polyamide (Nielsen et al., Science (1991) 254:1497) and phosphate residues may be replaced by ester groups such as diphosphate, alkyl, arylphosphonate and phosphorothioate esters. In addition, the 2′-hydroxyl group on ribonucleotides may be modified by including such groups as alkyl groups.

Probes of the invention are used for identifying and isolating putative orthosomycin-producing microorganisms, as capture or detection probes. Such capture probes are conventionally immobilized on a solid support, directly or indirectly, by covalent means or by passive adsorption. A detection probe is labeled by a detection marker selected from: radioactive isotopes, enzymes such as peroxidase, alkaline phosphatase, enzymes able to hydrolyze a chromogenic or fluorogenic or luminescent substrate, compounds that are chromogenic or fluorogenic or luminescent, nucleotide base analogs, and biotin.

Probes of the invention are used in any conventional hybridization technique, such as dot blot (Maniatis et al., Molecular Cloning: A Laboratory Manual (1982) Cold Spring Harbor Laboratory Press, Cold Spring Harbor, N.Y.), Southern blot (Southern, J. Mol. Biol. (1975) 98:503), northern blot (identical to Southern blot with the exception that RNA is used as a target), or the sandwich technique (Dunn et al., Cell (1977) 12:23). The latter technique involves the use of a specific capture probe and/or a specific detection probe with nucleotide sequences that at least partially differ from each other.

A primer is a probe of usually about 10 to about 40 nucleotides that is used to initiate enzymatic polymerization of DNA in an amplification process (e.g., PCR), in an elongation process, or in a reverse transcription method. Primers used in diagnostic methods involving PCR are labeled by methods known in the art.

As described herein, the invention also encompasses (i) a reagent comprising a probe of the invention for detecting and/or isolating putative orthosomycin-producing microorganisms; (ii) a method for detecting and/or isolating putative orthosomycin-producing microorganisms, in which DNA or RNA is extracted from the microorganism and denatured, and exposed to a probe of the invention, for example, a capture probe or detection probe or both, under stringent hybridization conditions, such that hybridization is detected; and (iii) a method for detecting and/or isolating putative orthosomycin-producing microorganisms, in which (a) a sample is recovered or derived from the microorganism, (b) DNA is extracted therefrom, (c) the extracted DNA is primed with at least one, and preferably two, primers of the invention and amplified by polymerase chain reaction, and (d) the amplified DNA fragment is produced.

It is understood that the embodiments described herein are for illustrative purposes only and that various modifications or changes in light thereof will be suggested to persons skilled in the art and are to be included within the spirit and purview of this application and scope of the appended claims. All publications, patents and patent applications cited herein are hereby incorporated by reference in their entirety for all purposes.

1. An isolated nucleic acid molecule comprising a nucleic acid sequence selected from any of:
(a) a nucleic acid encoding any of everninomicin open reading frames (ORFs) 1 to 49 (SEQ ID NOS: 2, 5 to 7, 9 to 21, 23 to 35, 37 to 46, 48, and 50 to 58);
(b) a nucleic acid encoding a polypeptide encoded by any of everninomicin open reading frames (ORFS) 1 to 49 (SEQ ID NOS: 2, 5 to 7, 9 to 21, 23 to 35, 37 to 46, 48, and 50 to 58); and
(c) a nucleic acid encoding a polypeptide which is at least 75% identical in amino acid sequence to a polypeptide encoded by any of everninomicin open reading frames (ORFs) 1 to 49 (SEQ ID NOS: 2, 5 to 7, 9 to 21, 23 to 35, 37 to 46, 48, and 50 to 58).

2. The isolated nucleic acid of claim 1, wherein said nucleic acid comprises a nucleic acid encoding at least two open reading frames (ORFs) selected from the group consisting of ORF 1 to ORF 49 (SEQ ID NOS: 2, 5 to 7, 9 to 21, 23 to 35, 37 to 46, 48, and 50 to 58).

3. The isolated nucleic acid of claim 2, wherein said nucleic acid comprises a nucleic acid encoding at least three open reading frames (ORFs) selected from the group consisting of ORF 1 to ORF 49 (SEQ ID NOS: 2, 5 to 7, 9 to 21, 23 to 35, 37 to 46, 48, and 50 to 58).

4. An isolated nucleic acid comprising a nucleic acid that hybridizes under stringent conditions to an open reading frame (ORF) of the everninomicin biosynthesis gene cluster and can substitute for the ORF to which it specifically hybridizes to direct the synthesis of an everninomicin.

5. The isolated nucleic acid of claim 4, wherein the isolated nucleic acid specifically hybridizes under stringent conditions to a nucleic acid encoding a polypeptide selected from the group comprising of ORF 1, ORF 2, ORF 3, ORF 4, ORF 5, ORF 6, ORF 7, ORF 8, ORF 9, ORF 10, ORF 11, ORF 12, ORF 13, ORF 14, ORF 15, ORF 16, ORF 17, ORF 18, ORF 19, ORF 20, ORF 21, ORF 22, ORF 23 and ORF 24 (SEQ ID NOS: 2, 5 to 7, 9 to 21, and 23 to 29).

6. The isolated nucleic acid of claim 4 wherein the nucleic acid specifically hybridizes under stringent conditions to a nucleic acid encoding a polypeptide selected from the group consisting of ORF 25, ORF 26, ORF 27, ORF 28, ORF 29, ORF 30, ORF 31, ORF 32, ORF 33, ORF 34, ORF 35, ORF 36, ORF 37, ORF 38, ORF 39, ORF 40, ORF 41, ORF 42, ORF 43, ORF 44, ORF 45, ORF 46, ORF 47, ORF 48 and ORF 49 (SEQ ID NOS 30 to 35, 37 to 46, 48 and 50 to 58).

7. The isolated nucleic acid of claim 5 wherein the isolated nucleic acid encodes a polypeptide selected from the group consisting of ORF 1, ORF 2, ORF 3, ORF 4, ORF 5, ORF 6, ORF 7, ORF 8, ORF 9, ORF 10, ORF 11, ORF 12, ORF 13, ORF 14, ORF 15, ORF 16, ORF 17, ORF 18, ORF 19, ORF 20, ORF 21, ORF 22, ORF 23 and ORF 24 (SEQ ID NOS: 2, 5 to 7, 9 to 21, and 23 to 29).

8. The isolated nucleic acid of claim 6 wherein the isolated nucleic acid encodes a polypeptide selected from the group consisting of ORF 25, ORF 26, ORF 27, ORF 28, ORF 29, ORF 30, ORF 31, ORF 32, ORF 33, ORF 34, ORF 35, ORF 36, ORF 37, ORF 38, ORF 39, ORF 40, ORF 41, ORF 42, ORF 43, ORF 44, ORF 45, ORF 46, ORF 47, ORF 48 and ORF 49 (SEQ ID NOS 30 to 35, 37 to 46, 48 and 50 to 58).

9. An isolated gene cluster comprising open reading frames encoding polypeptides sufficient to direct the synthesis of an everninomicin or an everninomicin analogue.

10. The isolated gene cluster of claim 9 wherein the gene cluster is present in a bacterium.

11. The isolated gene cluster of claim 9 wherein the gene cluster is present in E. coli strains DH10B having accession nos. IDAC 240101-1, IDAC 240101-2 and IDAC 240101-3.

12. An isolated polypeptide comprising a polypeptide sequence selected from any one of:
a) a polypeptide of open reading frames 1 to 49 (SEQ ID NOS: 2, 5 to 7, 9 to 21, 23 to 35, 37 to 46, 48, and 50 to 58); and
b) a polypeptide which is at least 75% identical in amino acid sequence to a polypeptide sequence of open reading frames (ORFs) 1 to 49 (SEQ ID NOS: 2, 5 to 7, 9 to 21, 23 to 35, 37 to 46, 48, and 50 to 58).

13. The polypeptide of claim 12, wherein said polypeptide is a polypeptide containing at least two open reading frames selected from open reading frames (ORFs)1 to 49 (SEQ ID NOS: 2, 5 to 7, 9 to 21, 23 to 35, 37 to 46, 48, and 50 to 58).

14. The polypeptide of claim 12, wherein said polypeptide is a polypeptide containing at least three open reading frames selected from open reading frames (ORFs) 1 to 49 (SEQ ID NOS: 2, 5 to 7, 9 to 21, 23 to 35, 37 to 46, 48, and 50 to 58).

15. The polypeptide of claim 12, wherein said polypeptide is a polypeptide containing at least three or more open reading frames selected from open reading frames 1 to 49 (SEQ ID NOS: 2, 5 to 7, 9 to 21, 23 to 35, 37 to 46, 48, and 50 to 58).

16. An expression vector comprising a nucleic acid of claim 1.

17. A host cell transformed with an expression vector of claim 16.

18. The host cell of claim 17, wherein the cell is transformed with an exogenous nucleic acid comprising a gene cluster encoding polypeptides sufficient to direct the assembly of an everninomicin or an everninomicin analogue.

19. A method of chemically modifying a biological molecule, said method comprising contacting a biological molecule that is a substrate for a polypeptide encoded by an everninomicin biosynthesis gene cluster open reading frame with a polypeptide encoded by an everninomicin biosynthesis gene cluster open reading frame whereby said polypeptide chemically modifies said biological molecule.

20. The method of claim 19 wherein said method comprises contacting said biological molecule with at least two different polypeptides encoded by everninomicin biosynthesis gene cluster open reading frames 1 to 49 (SEQ ID NOS: 2, 5 to 7, 9 to 21, 23 to 35, 37 to 46, 48, and 50 to 58).
